(19) United States
(12) Patent Application Publication
(10) Pub. No.: US 2026/0301867 A1
(43) Pub. Date: Oct. 1, 2026

(54) DEEP LEARNING METHODS FOR WHOLE GENOME SINGLE-CELL EPIGENETIC STATE DATA USING LONG-READ DNA METHYLATION SEQUENCE ANALYSIS

(71) Applicant: Petaomics, Inc., Austin, TX (US)

(72) Inventor: Paul M. Lizardi, London (GB)

(21) Appl. No.: 19/426,615

(22) Filed: Dec. 19, 2025

Related U.S. Application Data
(60) Provisional application No. 63/781,495, filed on Apr. 1, 2025.

(51) Int. Cl. G16B 25/10; G16B 30/10; G16B 40/30; G16B 45/00

(52) U.S. Cl. CPC G16B 25/10; G16B 30/10; G16B 40/30; G16B 45/00

(57) ABSTRACT

Methods for genome-wide DNA methylation analysis at single-cell resolution are provided. In some forms, biological samples to be analyzed contain multiple cell types, and individual cells are present in multiple regulatory states. The methods obtain single-cell epigenetic information including repetitive DNA elements, and provide full sets of 46 chromosomes corresponding to specific cell regulatory states. The methods include grouping assembly performed by alignment of long DNA sequences containing linearly contiguous DNA methylation information for entire chromosomes, guided by DNA methylation sequence information present in single-cell Methyl-seq data. Grouping assembly generates a multiplicity of distinct full sets of chromosome complements. The methods generate improved single-cell Methyl-seq data sets, augmented by additional information that accurately maps the DNA methylation states of all repetitive elements. The methods include data-acquisition and AI-based transformation of the acquired data.

### CROSS-REFERENCE TO RELATED APPLICATIONS

This application claims the benefit of and priority to U.S. Provisional Application No. 63/781,495 filed on Apr. 1, 2025, the contents of which is incorporated herein in its entirety.

### FIELD OF THE INVENTION

The disclosed invention is generally related to methods for sequence-specific identification of cell subtypes and lineages amongst a heterogeneous population of cells, specifically for the identification of the methylation status of chromosomal DNA.

### BACKGROUND OF THE INVENTION

The study of cell heterogeneity in tissues is of fundamental importance for progress in immunology research and for the emerging field of immune diagnostics. Identification and quantification of the individual components of a heterogeneous cell population in mammalian tissues remains a significant challenge, especially in the study of tissues and cells which include a large variety of morphologically and functionally distinct cell types. For example, human blood may contain more than 30,000 cell sub-populations. The study of cell heterogeneity continues to be a key subject of interest in medical research. Understanding individual components of a heterogeneous cell population represents an important unmet need in biological and clinical research.

Diverse cell populations are conventionally studied at multiple molecular and phenotypic levels, using a range of molecular characterization approaches including DNA sequencing, analysis of chromatin modifications, RNA expression profiling, and protein expression profiling (Satija and Shalek, 2014). However, molecular analysis, such as an RNA expression profile of a heterogeneous mixture of several thousands of cells provides an “average RNA expression profile,” which is not at all representative of the actual biological state of individual cells in the heterogeneous mixture.

To avoid the pitfalls of analyzing bulk cell populations, which often yield a blurred molecular profile of the “average” cell, single-cell molecular profiling strategies employ separation techniques to enable profiling of individual cells. For example, interest in molecular analysis of single cells has led to the development of highly advanced technologies for gene expression analysis at this level.

Advances in single-cell technologies have enabled comprehensive studies of cell heterogeneity and developmental dynamics across diverse biological systems. There are a variety of advanced protocols that enable profiling of transcription profiles, as exemplified by single-cell RNA sequencing (scRNA-seq). The most comprehensive understanding of cellular states at the single-cell level is achieved when it is possible to obtain multiple modalities of omics data from paired samples, such as RNA-seq and MTase-seq (chromatin accessibility). Optionally, three paired samples can be analyzed by RNA-seq, MTase-seq, and Methyl-seq (DNA methylation).

Single-cell Methyl-seq is a powerful tool for understanding epigenetic variation at the single-cell level. However, it faces significant challenges when attempting to generate DNA methylation information for loci harboring repetitive DNA elements. The key problem preventing the generation of DNA methylation information for DNA repeats is the impossibility of mapping each repetitive sequence unambiguously to its specific genomic coordinates when the DNA reads are short (150 to 250 bases). In the presence of stress or disease the methylation levels of repetitive DNA elements located at specific positions in the genome can be altered in ways that profoundly affect a cell's regulatory control. These important alterations in DNA methylation are not captured by single-cell Methyl-seq data sets, resulting in the absence of DNA methylation information for tens of thousands of DNA enhancer elements that contain repetitive DNA sequences. Further, existing “bulk” cell molecular profiling strategies do not provide an accurate profile of each individual cell, or even each cell type within a population/complex sample but instead yield a blurred molecular profile of the “average” cell within the population. These strategies do not, therefore enable a greater depth of characterization that is necessary for accurate diagnostic/prognostic or therapeutic approaches.

There remains a need for analytical approaches capable of yielding molecular profiles of the chromatin regulatory states of different cells in thousands of heterogeneous clinical samples, without the need to deploy single-cell analysis technologies at every step of the way. Such capabilities can be of great importance for analyzing large numbers of stored clinical samples, such as frozen tissues or frozen white cells, which often are not of suitable quality for single-cell analysis.

Effective methods to develop and effectively utilize long-read DNA methylation data have yet to be established, and there remains a lack of suitable methods to analyze the cytosine methylation status of heterologous cell populations within an organism.

Therefore, it is an object of the invention to provide methods for characterizing and quantifying DNA methylation data from one or more cells or subtypes from complex mixtures of cell subtypes, without necessarily isolating single cells prior to analysis.

It is a further object of the invention to provide methods for obtaining genome-wide information of DNA methylation states in complex tissue samples or blood samples at pseudo-single-cell resolution.

It is a further object of the invention to provide methods for obtaining single-cell epigenetic information that includes repetitive DNA elements.

It is a further object of the invention to provide methods for obtaining single-cell cell regulatory state information from complex tissues without the need to perform single-cell analysis of all types of samples.

It is a further object of the invention to provide methods for obtaining cell regulatory state information from complex tissues without the need to perform single-cell analysis of all types of samples.

### BRIEF SUMMARY OF THE INVENTION

Systems and methods that combine large-scale omics diverse datasets and pretrained transformers for developing foundation models have been established. Generative foundation models employing combinations of single-cell data and bulk tissue data for multi-omics analyses are provided.

The disclosed methods are useful for generating pseudo-single-cell epigenomic sequence data for a full chromosome complement, where the generated epigenomic sequence data can be representative of and resolved into different individual cell regulatory states. In some forms, the epigenomic sequence data can include a combination of DNA sequence data provided by long-read DNA methylation sequence data and single-cell-resolved DNA methylation sequence data grouped by cell regulatory state. In these forms, the single-cell-resolved DNA methylation sequence data constitutes cell-regulatory-state-resolved DNA methylation sequence data.

In some forms, the methods can use a Foundation model to generate pseudo-single-cell epigenomic sequence data for a full chromosome complement using only long-read DNA methylation sequence data generated from a newly obtained sample of cells, without the need to generate single-cell-resolved DNA methylation sequence data from the newly obtained sample of cells. In some forms, the generated epigenomic sequence data can be representative of and resolved into different individual cell regulatory states. In these forms, the generated epigenomic sequence data constitutes cell-regulatory-state-resolved epigenomic sequence data. In some forms, the Foundation model can be trained using pseudo-single-cell epigenomic sequence data for a full chromosome complement generated using a combination of DNA sequence data provided by long-read DNA methylation sequence data and single-cell-resolved DNA methylation sequence data grouped by cell regulatory state.

In some forms, the generated pseudo-single-cell epigenomic sequence data can be used to generate other forms of pseudo-single-cell full genome epigenomic data such as pseudo-single-cell RNA-seq data and pseudo-single-cell ATAC-seq data.

Disclosed are compositions, methods, and systems for obtaining genome-wide information of DNA methylation states in a complex biological sample at pseudo-single-cell resolution. In some forms, the methods involve generating pseudo-single-cell-resolved cell regulatory states from a test sample including a heterogenous population of cells. In some forms, the methods involve generating cell-regulatory-state-resolved epigenomic sequence data from a test sample including a heterogenous population of cells.

In some forms, the methods can include mapping single-cell-resolved DNA methylation sequence data to a set of epi-Haplotype Chromosomes (eHCs) to thereby group the different eHCs in the set of eHCs into single-cell-resolved subsets of eHCs (epi-Haplotype Genomes). In some forms, each epi-Haplotype Genome (eHG) can correspond to the full chromosome complement of respective different single cells represented in the single-cell-resolved DNA methylation sequence data. In some forms, the single-cell-resolved DNA methylation sequence data can be divided into sets of reads, where the reads in each set of reads correspond to respective different single cells represented in the single-cell-resolved DNA methylation sequence data. In some forms, the single-cell-resolved DNA methylation sequence data can be divided into sets of reads, where the reads in each set of reads correspond to respective different cell regulatory state represented in the single-cell-resolved DNA methylation sequence data. In these forms, the single-cell-resolved DNA methylation sequence data constitutes cell-regulatory-state-resolved DNA methylation sequence data.

In some forms, the eHCs in the set of eHCs can each be single-chromosome-resolved DNA methylation sequence data generated from assembly of long-read DNA methylation sequence data derived from one or more first sample(s). In some forms, the single-cell-resolved DNA methylation sequence data can be derived from one or more second sample(s). In some forms, the long-read DNA methylation sequence data can constitute a first omics data set and the single-cell-resolved DNA methylation sequence data can constitute a second omics data set. In some forms, each first sample in the one or more first sample(s) can constitute a pair of samples with one of the second sample(s) in the one or more second sample(s). In some forms, the first and second samples of a given pair of samples can be derived from the same source and include overlapping cell types and/or cell lineages. In some forms, the samples can include samples from multiple cell types, multiple cell lineages, multiple tissues, and/or multiple organs from a single individual or from multiple individuals. In some forms, the assembly of long reads of the long-read DNA methylation sequence data can include alignment of the long reads to form sets of continuously-aligned long reads where the alignment is based on linearly contiguous DNA methylation information in the long reads. In some forms, following alignment, each set of continuously-aligned long reads that makes up a full chromosome can constitute an eHC for that chromosome.

In some forms, the mapping of single-cell-resolved DNA methylation sequence data can include scoring of sequence alignment between each eHC in the set of eHCs against single-cell-resolved reads in the second omics data set. In some forms, the sequence alignment of single-cell-resolved reads containing repetitive DNA sequences are not scored. In some forms, the mapping of single-cell-resolved DNA methylation sequence data can include alignment of a pool of DNA methylation sequence data from the first omics data set with DNA methylation sequence data from the second omics data set. In some forms, the pool of DNA methylation sequence data can include one or more of the eHCs. In some forms, the DNA methylation sequence data from the second omics data set can include one or more sets of single-cell-resolved reads. In some forms, the single-cell-resolved reads in a given set of single-cell-resolved reads can be derived from an individual cell regulatory state. In some forms, the single-cell-resolved reads in a given set of single-cell-resolved reads can be derived from a single cell. In some forms, DNA methylation sequence data from the second omics data set can include defined genomic coordinates for the single-cell-resolved reads in the set(s) of single-cell-resolved reads except for the single-cell-resolved reads containing repetitive DNA sequences.

In some forms, the mapping of single-cell-resolved DNA methylation sequence data can include alignment of single-cell-resolved reads in the second omics data set with methylated DNA nucleotides in the eHCs except for the single-cell-resolved reads containing repetitive DNA sequences. In some forms, the alignment can include aligning single-cell-resolved reads from the second omics data set with each of the eHCs except for the single-cell-resolved reads containing repetitive DNA sequences and scoring the sequence alignment of the aligned single-cell-resolved reads with the eHCs.

In some forms, the grouping of eHCs into the eHGs can include selecting the eHC yielding the best aggregate sequence alignment score for each aligned class of single-cell-resolved reads and grouping the best scoring eHCs into a set of eHCs that constitute the eHG for the aligned class of single-cell-resolved reads. In some forms, the aligned class of single-cell-resolved reads correspond to a single cell regulatory state. In some forms, the aligned class of single-cell-resolved reads correspond to a single cell regulatory state and to multiple single cells representing that cell regulatory state. In some forms, the class of single-cell-resolved reads corresponds to a single cell regulatory state. In some forms, the different aligned classes of single-cell-resolved reads constitute cell-regulatory-state-resolved reads.

In some forms, segments of eHCs to which single-cell-resolved reads containing repetitive DNA sequences were not aligned can be assigned as the locus-specific DNA methylation states for those segments, thereby assigning to the corresponding single-cell-resolved data set the locus-specific DNA methylation states for all repetitive DNA sequences in the eHCs.

In some forms, the assembly of long-read DNA methylation sequence data can further involve storing the eHCs in a structured data format such as a first array. In some forms, the eHCs are stored within the first array, such as a first matrix. In some forms, the mapping of single-cell-resolved DNA methylation sequence data can further involve storing the eHGs in a structured data format such as a second array. In some forms, the eHGs are stored within the second array, such as a second matrix. The arrays containing the eHCs and eHGs can have the same or different structure and/or dimensions. In some forms, the second matrix can include the same structure and dimensions as the first matrix. In some forms, DNA methylation sequence data from both the first and second omics data sets can be mapped to the same eHGs except that DNA methylation sequence data from the second omics data set containing repetitive DNA sequences are not mapped.

In some forms, the assigning can further involve storing a combination of assigned single-cell-resolved reads and assigned segments of eHCs in a third array, which in some forms is a third matrix. In some forms, the third matrix can include long-read DNA methylation sequence data from the first omics data set(s) and corresponding single-cell-resolved DNA methylation sequence data from the second omics data set. In some forms, each assigned segment of eHC can be stored in an empty matrix position in the third matrix corresponding to the single-cell-resolved DNA methylation sequence data.

In some forms, each first sample can include a first portion of a biological sample of cells and/or tissue obtained from a subject and each second sample can include a second portion of the same biological sample of cells and/or tissue obtained from the subject. In some forms, the biological sample from which each pair of samples are taken can be different for each pair of samples. In some forms, at least one of the biological samples can be a complex biological sample. In some forms, each of the biological samples can be a complex biological sample. In some forms, the samples can include samples from multiple cell types, multiple cell lineages, multiple tissues, and/or multiple organs from a single individual or from multiple individuals.

In some forms, the long reads of the long-read DNA methylation sequence data in the first omics data set can include about 3,000 to about 100,000 nucleotides, inclusive. In some forms, the single-cell-resolved reads of the single-cell-resolved DNA methylation sequence data in the second omics data set can include about 50 to about 600 nucleotides, inclusive.

In some forms, the first omics data set can include sequencing data with a depth of coverage of more than 300, preferably of more than 600, inclusive, for each nucleotide position in the genome that is sequenced.

In some forms, the first and/or second omics data set(s) can include M5C modification data. In some forms, the M5C modification data can include a complete data set for all methylcytosines within each read in the first and/or second omics data set(s).

In some forms, the first and/or second omics data set(s) can include M6A modification data (Conti, et al., Elife. 2025 Apr. 7; 13: RP101626.) In some forms, the M 6A modification data can include a complete data set for all methyladenosines within each read in the first and/or second omics data set(s).

In some forms, the first and/or second omics data set(s) can include M5C methylation information as well as M6A modification data derived from the treatment of cells in a biological sample with a non-specific methylase, generating M6A base modifications at accessible deoxy-adenines in chromatin (Vollger, et al., bioRxiv [Preprint]. 2025 Jun. 2: 2024.06.14.599122.) In some forms, the first and/or second omics data set(s) can include M5C modification data and sequences that include bases with M6A modifications. In some forms, the M5C modification data and sequences that include bases with M6A modifications can include a complete data set for all methylcytosines and all methyladenosines within each read. In some forms, the first and/or second omics data set(s) can further include 5-hydroxy-methylCytosine modifications (Halliwell, et al., Commun Biol. 2025 Feb. 15; 8(1): 243).

In some forms, the first omics data set can be generated via OXFORD NANOPORE TECHNOLOGIES™ sequencing. In some forms, the OXFORD NANOPORE TECHNOLOGIES™ sequencing can be performed using a high-throughput, real-time DNA and RNA sequencing device such as an Oxford OXFORD NANOPORE TECHNOLOGIES™ PROMETHION™ DNA sequencer. In other forms, the first omics data set can be generated via Pacific Biosciences REVIO™ SPRQ™-Nx long read DNA sequencing platform that enables long read sequencing with accurate reporting of M5C, hydroxy-M5C and M6A base modifications using SPRQ™ chemistry. In some forms, the second omics data set can be generated by single-cell Methyl-seq.

In some forms, the assembly of long-read DNA methylation sequence data can involve de novo DNA sequence assembly of long reads in the first omics data set into the eHCs. In some forms, the eHCs can constitute full-length methylated DNA sequence of entire chromosomes, sometimes including the centromeres. In some forms, the generation of chromosome assemblies that span the centromere will be successful for a few chromosomes. In other forms, the centromere is too long to permit the sequence assembly across the entire centromere. In some forms, the assembly of long-read DNA methylation sequence data can be performed using software for haplotype-aware de novo assembly of diploid genomes from long reads. In some forms, the software can include PHASEBOOK software. In some forms, the mapping of single-cell-resolved DNA methylation sequence data can be performed using available alignment software such as MAFFT, Minimap2, or MUMMEr4.

In some forms, each eHG can correspond to the full chromosome complement of a cell regulatory state represented in the single-cell-resolved DNA methylation sequence data. In these forms, the single-cell-resolved DNA methylation sequence data constitutes cell-regulatory-state-resolved DNA methylation sequence data. In some forms, the full chromosome complement of each cell regulatory state can include repetitive DNA sequences that are differentially methylated in stressed or diseased cells. In some forms, the cell regulatory states can include one or more disease states.

In some forms, prior to production of the first and second omics data sets, the first and second samples or their source sample can be depleted of the most highly abundant cell subtypes. The resulting first and second sample(s) can be referred to as depleted first and second samples.

In some forms, one or more additional first and second omics data sets can be produced from one or more additional first and second sample derived from the same source as the first and second samples of a given pair of samples. In some forms, the additional first and second samples or their source sample can be depleted of the most highly abundant cell subtypes. The resulting first and second sample(s) can be referred to as depleted first and second samples.

In some forms, long-read DNA methylation sequence data derived from one or more depleted first samples can result in a larger proportion of the eHCs being derived from cell types and/or cell lineages present at low frequency in undepleted samples as compared to when long-read DNA methylation sequence data derived from one or more undepleted first samples are assembled into eHCs. In some forms, single-cell-resolved DNA methylation sequence data derived from one or more depleted second samples can result in a larger proportion of the eHCs being derived from cell types and/or cell lineages present at low frequency in undepleted samples as compared to when single-cell-resolved DNA methylation sequence data derived from one or more undepleted second samples are aligned to eHCs to group the eHCs into eHGs.

In some forms, the methods can further involve formulating the eHCs as a training set for a machine learning architecture. In some forms, the architecture can include a foundation model and a transformer. In some forms, the model and transformer each can include encoding systems for training based on the training set of eHCs and corresponding second omics data sets. In some forms, the model and transformer can learn the sequence relationships among all of the eHGs.

In some forms, the methods can further involve generating, using the machine learning architecture, one or more further eHCs from one or more further omics data set(s) that each include further long-read DNA methylation sequence data derived from one or more further sample(s). In some forms, the machine learning architecture can generate a plurality of eHGs each corresponding to the full chromosome complement of respective different single cells represented in the further long-read DNA methylation sequence data of the further omics data set(s). In some forms, each eHIG can represent a cell regulatory state for a single cell in the further sample(s).

Also disclosed are methods involving analyzing a test omics data set derived from a test sample using a deep learning (DL) model and assigning cell regulatory states to cells in the test sample without a need for analyzing single-cell omics data from the test sample. In some forms, the method can further involve displaying the assigned cell regulatory states on a graphical user interface in real-time. In some forms, the assigned cell regulatory states can be displayed on a graphical user interface within 1, 2, 3, 4, 5, 10, 15, 20, or no more than 30 minutes after receiving the eHC-assembled test omics data set.

In some forms, the DL model can be trained on one or more first omics data set(s) from one or more first sample(s) and one or more second omics data set(s) from one or more second sample(s). In some forms, each first sample in the one or more first sample(s) can constitute a pair of samples with one of the second sample(s) in the one or more second sample(s). In some forms, the first and second samples of a given pair of samples can be derived from the same source and include overlapping cell types and/or cell lineages. In some forms, the test sample can be derived from the same type of source as the source of at least one of the pairs of first and second samples.

In some forms, training the DL model can include the DL model learning relationships among the first and second omics data sets. In some forms, during training the DL model can learn and associate subsets of data within the first and second omics data sets as belonging to a single cell. In some forms, during training the DL model can learn and associate subsets of data within the first and second omics data sets as belonging to an individual cell regulatory state.

In some forms, the methods can further involve fine-tuning the DL model with one or more additional first and second omics data sets relevant for one or more of the cell regulatory states of the source of the samples. In some forms, the additional first and second omics data sets can be produced from one or more additional first and second sample derived from the same source as the first and second samples of a given pair of samples. In some forms, the additional first and second samples or their source sample can be depleted of the most highly abundant cell subtypes. The resulting additional first and second samples can be referred to as depleted first and second samples.

In some forms, the DL model can be a foundation model. In some forms, the DL model can be a language model. In some forms, the DL model can include a Hyena architecture, or a transformer architecture. In some forms, the DL model can include a HyenaDNA architecture, or a StripedHyena architecture.

In some forms, the DL model can tokenize data at molecular scale resolution. In some forms, the DL model can tokenize data at single-nucleotide resolution.

In some forms, the test omics data set from the test sample can include long-read DNA methylation sequencing data of the test sample. In some forms, the first omics data set from the first sample(s) can include long-read DNA methylation sequencing data of the first sample(s). In some forms, the second omics data set from the second sample(s) can include single-cell-resolved DNA methylation sequencing data of the second sample(s).

In some forms, the test omics data set from the test sample can include DNA having between 3,000 and 100,000 nucleotides. In some forms, the first omics data set from the first sample(s) can include DNA having between 3,000 and 100,000 nucleotides.

In some forms, the second omics data set derived from the second sample(s) can include single-cell-resolved DNA methylation sequence data. In some forms, the second omics data set from the second sample(s) can include DNA having between 100 and 600 nucleotides.

In some forms, assigning cell regulatory states to cells in the test sample without a need for analyzing single-cell omics data from the test sample can involve querying the trained DL model with a test long-read DNA methylation sequence data set derived from the test sample and assigning epi-Haplotype Chromosomes (eHCs) to cell regulatory states. In some forms, the eHCs can be single-chromosome-resolved DNA methylation sequence data generated from assembly of the test long-read DNA methylation sequence data followed by a query that is processed by the trained DL model.

In some forms, one or more of the omics data sets can include a modality selected from the group consisting of single-cell transcription (RNA-seq) profiles, single-cell transposase chromatin accessibility profiles (ATAC-seq) profiles, single-cell M6A-MTase chromatin accessibility (MTase-seq) profiles, single-cell genome-wide DNA methylation (Methyl-seq) profiles, single-cell chromosome conformation capture (Hi-C-seq) profiles, bulk transcription profiles generated from whole-tissue RNA (bulk-RNA-seq) profiles, and bulk Deep long-Read DNA methylation profiles.

In some forms, one or more of the omics data sets can include a modality where the modality can include DNA sequences with genomic coordinates. In some forms, the DNA sequence data can further include M5C modification data. In some forms, the DNA sequence data can further include sequences including bases with M6A modifications. In some forms, the DNA sequence data can further include M5C modification data and sequences including bases with M6A modifications. In some forms, the DNA sequence data can further include 5-hydroxy-methylCytosine modifications.

In some forms, one or more of the samples can include a heterogeneous mixture including a multiplicity of different cell types. In some forms, two or more cells of the same cell type can include two or more different cell regulatory states.

In some forms, one or more of the samples can include a peripheral blood sample from a subject. In some forms, the peripheral blood sample can include a multiplicity of leucocyte cells. In some forms, two or more leucocyte cells can include two or more different cell regulatory states.

In some forms, one or more of the samples can be derived from a biological sample at different time points. In some forms, one or more of the samples can be derived from a biological sample before and after treatment of the biological sample with an active agent. In some forms, the active agent can include a therapeutic agent, or a toxin.

In some forms, one or more of the samples can be derived from a biological sample before and after being depleted of all cells that include one or more cell subtype(s). In some forms, the depleted cells can include an abundant cell type in the first sample that are depleted from the second sample.

Also disclosed are non-transitory computer-readable mediums. In some forms, the computer-readable mediums can include executed instructions stored thereon. In some forms, the executed instructions can be executed by a processor to perform a method that can involve using a deep learning (DL) model to assign, in real time, cell regulatory states to cells in one or more test sample(s) without a need for analyzing single-cell omics data from the test sample(s).

In some forms, the DL model can be trained on at least one or more first omics data set(s) from one or more first sample(s) and one or more second omics data set(s) from one or more second sample(s). In some forms, each first sample in the one or more first sample(s) can constitute a pair of samples with one of the second sample(s) in the one or more second sample(s). In some forms, the first and second samples of a given pair of samples can be derived from the same source and can include overlapping cell types and/or cell lineages. In some forms, the test sample can be derived from the same type of source as the source of at least one of the pairs of first and second samples. In some forms, the DL model can be operably linked to a graphical user interface. In some forms, one or more of the samples can include a heterogenous population of cells.

### BRIEF DESCRIPTION OF THE DRAWINGS

FIGS. 1A-1B are flow-charts showing features and logic of an example of the disclosed methods. Multiple eHap chromosomes (eHCs) are generated by sequence assembly of long DNA methylation reads. Each eHap chromosome is compared to distinct sets of single-cell short sequences generated by Methyl-seq data, using pairwise alignment. After alignment, those subsets of 46 eHaps with the best aggregate scores are grouped and assigned to each single-cell regulatory state defined by the Methyl-seq data. The final outcome of this process is to assign each of the correctly mapped repetitive elements present in the eHap data to the corresponding single-cell data set. Each repetitive DNA sequence has a defined DNA methylation pattern, representative of a unique cell regulatory state, which is now added to the single-cell Methyl-seq data set, resulting in a dramatic enhancement of the information content of the single-cell data.

FIG. 2 is a flow chart showing features and logic of an example of the disclosed methods using a Foundation model. Two modalities of DNA methylation epigenomic sequence data, including bulk long reads and single-cell Methyl-seq short reads are obtained from PAIRED samples, in order to generate multiple sets of full chromosome complements representative of and resolved into different individual cell regulatory states. The resulting structured data including distinct groups of 46 chromosome complements is used to train a Foundation model. This is followed by use of the trained Foundation model to generate pseudo-single-cell epigenomic sequence data for full chromosome complements representative of and resolved into different individual cell regulatory states, from only long-read DNA methylation sequence data generated from a newly obtained sample of cells, without the need to generate single-cell-resolved Methyl-seq DNA methylation sequence data from the newly obtained sample of cells.

FIG. 3 is a flow chart showing features and logic of an example of the disclosed methods using a trained Foundation model to generate pseudo-single-cell epigenomic sequence data (generated by, for example, the method of FIG. 2) followed by use of a trained BABEL DL model to generate pseudo-single-cell RNA-seq data or pseudo-single-cell ATAC-seq data.

### DETAILED DESCRIPTION OF THE INVENTION

### A. Definitions

As used herein, “enrich” and “enrichment” refer to an increase in the proportion of a component relative to other components present or originally present. In the context of nucleic acids, enrichment of nucleic acids in a sample refers to an increase in the proportion of the nucleic acids in the sample relative to other molecules in the sample. “Selective enrichment” is enrichment of particular components relative to other components of the same type. In the context of nucleic acid fragments, selective enrichment of a particular nucleic acid fragment refers to an increase in the proportion of the particular nucleic acid fragment in a sample relative to other nucleic acid fragments present or originally present in the sample. The measure of enrichment can be referred to in different ways. For example, enrichment can be stated as the percentage of all of the components that is made up by the enriched component. For example, particular nucleic acid fragments can be enriched in an enriched nucleic acid sample to at least 90% of the enriched nucleic acid sample.
As used herein, the term “Partial depletion” refers to the removal of less than the total number of an element or components present within a mixture. For example, a partially-depleted population of cells can contain less cells than the equivalent “non-depleted population from which the depleted population is derived. The term “partially depleted sample” refers to a sample which is depleted of one or more specific recognized elements, as compared to the equivalent non-depleted sample from which the depleted sample is derived. Typically, a partially depleted sample is depleted of one or more specific recognized elements, as compared to the equivalent non-depleted sample from which the partially-depleted sample is derived.
As used herein, “CpG” site or “CG” site refers to a cytosine-guanine dinucleotide, such as a region of a nucleic acid where a cytosine nucleotide occurs next to a guanine nucleotide in the linear sequence of bases along its length. Cytosines in CpG dinucleotides can be methylated by addition of a methyl group to form 5-methylcytosine, for example, by the activity of intra-cellular DNA methyltransferases enzymes. In mammals, methylating the cytosine within or near a gene can change its expression.
As used herein, “nucleic acid fragment” refers to a portion of a larger nucleic acid molecule. A “contiguous nucleic acid fragment” refers to a nucleic acid fragment that represents a single, continuous, contiguous sequence of the larger nucleic acid molecule. A “naturally occurring nucleic acid fragment” refers to a nucleic acid fragment that represents a single, continuous, contiguous sequence of a naturally occurring nucleic acid sequence.
As used herein, “DNA fragment” refers to a portion of a larger DNA molecule. A “contiguous DNA fragment” refers to a DNA fragment that represents a single, continuous, contiguous sequence of the larger DNA molecule. A “naturally occurring DNA fragment” refers to a DNA fragment that represents a single, continuous, contiguous sequence of a naturally occurring DNA sequence.
As used herein, “naturally occurring” refers to a molecule that has the same structure or sequence as the corresponding molecule as it exists in nature. A naturally occurring molecule or sequence can still be considered naturally occurring when it is coupled to or incorporated into another molecule or sequence.
As used herein, “nucleic acid sample” refers to a composition, such as a solution, that contains or is suspected of containing nucleic acid molecules. An “enriched nucleic acid sample” is a nucleic acid sample in which nucleic acids, particular nucleic acid fragments, or a combination thereof, are enriched.
As used herein, “DNA sample” refers to a composition, such as a solution, that contains or is suspected of containing DNA molecules. An “enriched DNA sample” is a DNA sample in which DNA, particular DNA fragments, or a combination thereof, are enriched.
As used herein, “denatured nucleic acid” or “denatured DNA” refers to a nucleic acid that is denatured relative to a prior existing “native” or “non-denatured” state. For example, double-stranded nucleic acids, such as naturally-occurring dsDNA strands are completely denatured when separated into two corresponding single-stranded nucleic acid strands.

Denaturation of nucleic acids can occur by chemical or physical means, such as exposure to salts or increased temperatures above the meeting temperature of the dsDNA, or by interaction of dsDNA with a denaturing molecule, such as an antibody or enzyme.

Denaturation can be partial, for example, resulting in partially or substantially denatured DNA, or complete, resulting in completely denatured DNA. Nucleic acid that has never been subjected to partial or complete denaturation is referred to as “never-denatured nucleic acid,” such as never-denatured dsDNA.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.
As used herein, the term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein. As used herein, the term “nucleotide analog” refers to a nucleotide which contains some type of modification to the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.
As used herein, the term “nucleotide substitute” refers to a nucleotide molecule having similar functional properties to nucleotides, but which does not contain a phosphate moiety. An exemplary nucleotide substitute is peptide nucleic acid (PNA). Nucleotide substitutes are molecules that will recognize nucleic acids in a Watson-Crick or Hoogsteen manner, but which are linked together through a moiety other than a phosphate moiety.

Nucleotide substitutes are able to conform to a double helix type structure when interacting with the appropriate target nucleic acid. There are many varieties of these types of molecules available in the art and available herein. It is also possible to link other types of molecules (conjugates) to nucleotides or nucleotide analogs to enhance for example, interaction with DNA. Conjugates can be chemically linked to the nucleotide or nucleotide analogs. Exemplary conjugates include but are not limited to lipid moieties such as a cholesterol moiety (Letsinger, et al., Proc. Natl. Acad. Sci. USA, 1989,86, 6553-6556). There are many varieties of these types of molecules available in the art and available herein.
As used herein, the terms “oligonucleotide” or a “polynucleotide” are synthetic or isolated nucleic acid polymers including a plurality of nucleotide subunits.

The terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed oligonucleotides, nucleotide analogs, or nucleotide substitutes thereof and proteins disclosed herein, is through defining the variants and derivatives in terms of homology to specific known sequences. This identity of particular sequences disclosed herein is also discussed elsewhere herein. In general, variants of oligonucleotides, nucleotide analogs, or nucleotide substitutes thereof and proteins disclosed herein typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level. Another way of calculating homology can be performed by published algorithms. Optimal alignment of sequences for comparison can be conducted by the local homology algorithm of Smith and Waterman Adv. Appl. Math. 2: 482(1981 ), by the homology alignment algorithm of Needleman and Wunsch, J. MOL Biol. 48: 443(1970 ), by the search for similarity method of Pearson and Lipman, Proc. Natl. Acad. Sci. U.S.A. 85: 2444(1988 ), by computerized implementations of these algorithms (GAP, BESTFIT, FASTA, and TFASTA in the Wisconsin Genetics Software Package, Genetics Computer Group, 575 Science Dr., Madison, WI), or by inspection. The same types of homology can be obtained for nucleic acids by for example the algorithms disclosed in Zuker, M. Science 244: 48-52, 1989, Jaeger et al. Proc. Natl. Acad. Sci. USA 86: 7706-7710, 1989, Jaeger et al. Methods Enzymol. 183: 281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).
As used herein, the term “subject” includes, but is not limited to, animals, plants, bacteria, viruses, parasites and any other organism or entity. The subject can be a plant. The subject can be an animal, such as a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. A cell can be in vitro. Alternatively, a cell can be in vivo and can be found in a subject. A “cell” can be a cell from any organism including, but not limited to, a mammal, such as a human.
The term “modality” as used herein, refers to a type of data, i.e., relating to a specific biological characteristic of a cell or organism. In a first exemplary form, a modality is or includes DNA methylation. In a second exemplary form, a modality is or includes chromatin accessibility.
The term “Long-read DNA methylation sequencing”, as used herein, refers to long-read third-generation-sequencing technologies that provide nucleic acid sequences of greater than about 300 residues in length, up to several thousand residues in length.
The term “paired sample” as used herein, refers to a set of sample(s), or to data obtained from a set of samples, whereby the sample is or includes material obtained from the same source. For example, in some forms, a paired sample includes a first sample and a second sample derived from the same bulk tissue or cells. In some forms, a paired sample includes a first sample of bulk cells and/or tissue obtained from a subject, and a second sample of individual cell(s) obtained from the same subject.
The term “Transformer” as used herein, refers to a Deep learning architecture based on parallel attention mechanism.
The term “Attention mechanism” as used herein, refers to a data-adaptive neural network component that dynamically focuses on the relevant information in the input to compute the output.
The term “Self-attention” as used herein, refers to a type of attention mechanism that focuses solely on the relationships between the input embeddings, unlike traditional attention mechanisms that focus on the relationship between the input and output embeddings as well.
The terms “Key”, “query”, and “value” as used herein, refer to components of the attention mechanism in transformer models. Queries are elements for which the model seeks relevant information. Keys are compared to queries to produce attention scores and values are the actual content that the model retrieves, which is finally weighted based on the query-key comparison.
The term “Multihead attention” as used herein, refers to a neural network composed of multiple attention mechanisms (heads), each with a separate set of parameters.
The term “Foundation model” as used herein, refers to a machine learning model trained on a large quality of data that can be effectively adapted to a wide range of downstream tasks.
The terms “RNA-seq” and “transcription profile” as used herein, refer to a technique that uses next-generation sequencing to reveal the presence and quantity of RNA molecules in a biological sample, providing a snapshot of gene expression in the sample, also known as transcriptome. Single-cell RNA-seq (scRNA-seq) is RNA-seq performed on or providing information on a single cell.
The terms “Assay for Transposase-Accessible Chromatin sequencing”, or “ATAC-seq” and “transposase chromatin accessibility”, as used herein, refer to a tool to elucidate the cellular epigenetic landscape by unveiling the active regulatory, or “open chromatin,” regions that control gene expression. Single-cell ATAC-seq (scATAC-seq) is ATAC-seq performed on or providing information on a single cell.
The terms “M6A-MTase chromatin accessibility” or “MTase-seq” as used herein, refer to a chromatin accessibility assay. Single-cell MTase-seq is MTase-seq performed on or providing information on a single cell.
The term “Methyl-seq”, “bisulfite sequencing” or “genome-wide DNA methylation” as used herein, refer to a method for studying methylation patterns in DNA samples. Methylation is an epigenetic modification that involves transferring a methyl group to nucleotides, primarily cytosines. Single-cell Methyl-seq is Methyl-seq performed on or providing information on a single cell.
The term “Hi-C-seq” or “chromosome conformation capture” as used herein, refer to single-cell Hi-C-seq is Hi-C-seq performed on or providing information on a single cell.
The term “bulk RNA-seq” as used herein, refers to a technique that uses next-generation sequencing to reveal the presence and quantity of RNA molecules in a bulk biological sample, providing a snapshot of gene expression in the sample, also known as transcriptome.
The term “bulk Deep Long-Read DNA methylation” as used herein, refers to methods for whole genome sequencing that generates de-novo DNA sequence assemblies including the full length of entire chromosomes, with the genomic assembly sometimes extending across the centromere. The whole-chromosome de-novo sequenced assemblies obtained from a bulk DNA sample extracted from a mixture including different cell types will resolve into many classes of epi-Haplotypes. Exemplary Deep Long-Read DNA methylation analyses are carried out using the OXFORD NANOPORE TECHNOLOGIES™ PROMETHION™ system. The term “Deep” typically indicates that the PROMETHION™ system is used to generate hundreds of reads from every nucleotide sequence position in the genome.
The term “BABEL” as used herein, refers to a deep learning method that translates between the transcriptome and chromatin profiles of a single cell. Leveraging an interoperable neural network model, BABEL can predict single-cell expression directly from a cell's scATAC-seq and vice versa after training on relevant data. This makes it possible to computationally synthesize paired multi-omic measurements when only one modality is experimentally available.
The term “Single-cell Generative Pre-trained Transformer (scGPT)” as used herein, refers to a foundation model for single-cell biology based on a generative pretrained transformer on single-cell sequencing data.
As used herein, “single-cell-resolved,” in the context of sequences, sequence data, and omics data, indicates that the sequences, sequence data, or omics data is identified and identifiable as relating to or as derived from individual cells. For example, single-cell-resolved DNA methylation sequence data is DNA methylation sequence data that is tagged or otherwise identifiable as relating to or as derived from individual cells. A given set of DNA methylation sequence data can include DNA methylation sequence data from many individual cells, but the data in such a set that relates to or derived from each of those individual cells can be identified (i.e., resolved) uniquely within the set. Single-cell RNA-seq (scRNA-seq) and single-cell ATAC-seq (scATAC-seq) are examples of methods that generate single-cell-resolved sequence data.
The term “haplotype” refers to a physical grouping of sequences, variants, polymorphisms, epigenomic features, etc. that are inherited together. A chromosome is generally the largest physical structure by which sequences, variants, polymorphisms, epigenetic features, etc. that are inherited together. Sub-chromosomal haplotypes are partial chromosomal physical groupings of sequences, variants, polymorphisms, epigenomic features, etc. that are inherited together that correspond to only a part of a chromosome.
The term “epi-haplotype” or “eHap” as used herein refers to a collection of haplotype-resolved epigenomic data or maps. These maps take into account both the genetic sequence of the haplotype and the associated DNA methylation that can influence the expression of genes in different cell types. The term highlights the interplay between the genetic code (haplotype) and the epigenome (the highly variable spectrum of epigenetic marks) in determining cell phenotype or disease susceptibility. Haplotype-resolved in the context of a set of epigenomic data or maps indicates that the set epigenomic data includes epigenomic data or maps related to different individual haplotypes and that the epigenomic data or maps in such a set that relate to each of those individual haplotypes can be identified (i.e., resolved) uniquely within the set. The term eHap encompasses sub-chromosomal epi-haplotypes and full chromosomal epi-haplotype.
The term “epi-Haplotype Chromosome” (eHC) refers to the subtype of eHap that encompass an entire chromosome or a chromosome-identifying sub-chromosomal eHap. In this context, the term “chromosome-identifying” indicates that sub-chromosomal eHap is sufficient to identify the individual chromosome to which the eHap belongs when analyzed using single-cell-resolved sequences, sequence data, or omics data. In this context, the individual chromosome can be identified both as the chromosome in the full chromosome complement (e.g., in the 46 human chromosomes) and the particular chromosome in an individual cell from which the epigenomic data was produced. The sequence constituting an eHC represents sequences of a physical chromosome. Further, eHCs are single-chromosome-resolved sequence data.
The term “epi-Haplotype Genome” (eHG) refers to a set of eHCs that corresponds to the full chromosome complement of respective different single cells represented in the single-cell-resolved sequences, sequence data, or omics data. An eHG represents the entire chromosome complement of an individual single cell. Further, eHGs are sets of single-cell-resolved eHCs of a full chromosome complement of the cell (e.g., 46 eHCs).
As used herein, “cell-regulatory-state-resolved,” in the context of sequences, sequence data, and omics data, indicates that the sequences, sequence data, or omics data are identified and identifiable as relating to or as derived from individual cell regulatory states. For example, cell-regulatory-state-resolved DNA methylation sequence data is DNA methylation sequence data that is identifiable (such as by distinctive DNA methylation patterns) as relating to or as derived from individual cell regulatory states.
As used herein, the term “single-chromosome-resolved” in the context of sequences, sequence data, and omics data, indicates that the sequences, sequence data, or omics data is identified and identifiable as relating to or as derived from individual chromosomes from individual cells.

The single-cell data of the disclosed methods generally represents not an actual single cell, but a molecular descriptor of a subset of “almost identical” single cells (10 to 300 cells, for example), all being classified by bioinformatics as belonging to the exact same regulatory state. In such cases, a full-chromosome eHap or a sub-chromosomal eHap is determined by aggregate alignment scores to belong to a specific eHG, and said specific eHG, including all its eHC chromosomes or sub-chromosomal eHaps, is determined to belong to a “category” that includes identical single cells (but literally the same cell). As used herein, “single cell” refers to both actual single cells or to such a category of identical single cells, unless the context indicates or requires one meaning to the exclusion of the other meaning. This is made possible by the focus of the disclosed methods on methylation states, which are highly cell-state-relevant.

### B. General Description

In some forms, the methods can include mapping single-cell-resolved DNA methylation sequence data to a set of epi-Haplotype Chromosomes (eHCs) to thereby group the different eHCs in the set of eHCs into single-cell-resolved subsets of eHCs (epi-Haplotype Genomes). In some forms, each epi-Haplotype Genome (eHG) can correspond to the full chromosome complement of respective different single cells represented in the single-cell-resolved DNA methylation sequence data. In some forms, the single-cell-resolved DNA methylation sequence data can be divided into sets of reads, where the reads in each set of reads correspond to respective different cell regulatory states represented in the single-cell-resolved DNA methylation sequence data. In some forms, the single-cell-resolved DNA methylation sequence data can be divided into sets of reads, where the reads in each set of reads correspond to respective different single cells represented in the single-cell-resolved DNA methylation sequence data. In some forms, the single-cell-resolved DNA methylation sequence data can be divided into sets of reads, where the reads in each set of reads correspond to respective different cell regulatory states represented in the single-cell-resolved DNA methylation sequence data. In these forms, the single-cell-resolved DNA methylation sequence data constitutes cell-regulatory-state-resolved DNA methylation sequence data.

In some forms, the eHCs in the set of eHCs can each be single-chromosome-resolved DNA methylation sequence data generated from assembly of long-read DNA methylation sequence data derived from one or more first sample(s). In some forms, the single-cell-resolved DNA methylation sequence data can be derived from one or more second sample(s). In some forms, the long-read DNA methylation sequence data can constitute a first omics data set and the single-cell-resolved DNA methylation sequence data can constitute a second omics data set. In some forms, each first sample in the one or more first sample(s) can constitute a pair of samples with one of the second sample(s) in the one or more second sample(s). In some forms, the first and second samples of a given pair of samples can be derived from the same source and include overlapping cell types and/or cell lineages.

In some forms, the assembly of long reads of the long-read DNA methylation sequence data can include alignment of the long reads to form sets of continuously-aligned long reads where the alignment is based on linearly contiguous DNA methylation information in the long reads. In some forms, following alignment, each set of continuously-aligned long reads that makes up a full chromosome can constitute an eHC for that chromosome.

In some forms, the mapping of single-cell-resolved DNA methylation sequence data can include scoring of sequence alignment between each eHC in the set of eHCs against single-cell-resolved reads in the second omics data set. In some forms, the sequence alignment of single-cell-resolved reads containing repetitive DNA sequences are not scored. In some forms, the mapping of single-cell-resolved DNA methylation sequence data can include alignment of a pool of DNA methylation sequence data from the first omics data set with DNA methylation sequence data from the second omics data set. In some forms, the pool of DNA methylation sequence data can include one or more of the eHCs. In some forms, the DNA methylation sequence data from the second omics data set can include one or more sets of single-cell-resolved reads. In some forms, the single-cell-resolved reads in a given set of single-cell-resolved reads can be derived from an individual cell regulatory state. In some forms, the single-cell-resolved reads in a given set of single-cell-resolved reads can be derived from a single cell. In some forms, DNA methylation sequence data from the second omics data set can include defined genomic coordinates for the single-cell-resolved reads in the set(s) of single-cell-resolved reads except for the single-cell-resolved reads containing repetitive DNA sequences.

In some forms, the mapping of single-cell-resolved DNA methylation sequence data can include alignment of single-cell-resolved reads in the second omics data set with methylated DNA nucleotides in the eHCs except for the single-cell-resolved reads containing repetitive DNA sequences. In some forms, the alignment can include aligning single-cell-resolved reads from the second omics data set with each of the eHCs except for the single-cell-resolved reads containing repetitive DNA sequences and scoring the sequence alignment of the aligned single-cell-resolved reads with the eHCs.

In some forms, the grouping of eHCs into the eHGs can include selecting the eHC yielding the best aggregate sequence alignment score for each aligned class of single-cell-resolved reads and grouping the best scoring eHCs into a set of eHCs that constitute the eHG for the aligned class of single-cell-resolved reads. In some forms, the aligned class of single-cell-resolved reads correspond to a single cell regulatory state and to multiple single cells representing that cell regulatory state. In some forms, the class of single-cell-resolved reads corresponds to a single cell regulatory state. In some forms, the different aligned classes of single-cell-resolved reads constitute cell-regulatory-state-resolved reads. In some forms, the multiple single cells representing that cell regulatory state include 2, 3, 4, 5, 6, 7, 8, 9, 10, 15, 20, 30, 40 50, 60, 70, 80, 90, 100, or more single cells.

In some forms, segments of eHCs to which single-cell-resolved reads containing repetitive DNA sequences were not aligned can be assigned as the locus-specific DNA methylation states for those segments, thereby assigning to the corresponding single-cell-resolved data set the locus-specific DNA methylation states for all repetitive DNA sequences in the eHCs.

In some forms, the assembly of long-read DNA methylation sequence data can further involve storing the eHCs in a structured data format such as a first array. In some forms, the eHCs are stored within the first array, such as a first matrix. In some forms, the mapping of single-cell-resolved DNA methylation sequence data can further involve storing the eHGs in a structured data format such as a second array. In some forms, the eHGs are stored within the second array, such as a second matrix. The arrays containing the eHCs and eHGs can have the same or different structure and/or dimensions. In some forms, the second matrix can include the same structure and dimensions as the first matrix. In some forms, DNA methylation sequence data from both the first and second omics data sets can be mapped to the same eHGs except that DNA methylation sequence data from the second omics data set containing repetitive DNA sequences are not mapped.

In some forms, the assigning can further involve storing a combination of assigned single-cell-resolved reads and assigned segments of eHCs in a third array, which in some forms is a third matrix. In some forms, the third matrix can include long-read DNA methylation sequence data from the first omics data set(s) and corresponding single-cell-resolved DNA methylation sequence data from the second omics data set. In some forms, each assigned segment of eHC can be stored in an empty matrix position in the third matrix corresponding to the single-cell-resolved DNA methylation sequence data.

In some forms, each first sample can include a first portion of a biological sample of cells and/or tissue obtained from a subject and each second sample can include a second portion of the same biological sample of cells and/or tissue obtained from the subject. In some forms, the biological sample from which each pair of samples are taken can be different for each pair of samples. In some forms, at least one of the biological samples can be a complex biological sample. In some forms, each of the biological samples can be a complex biological sample.

In some forms, the samples can include samples from multiple cell types, such as 2, 3, 4, 5, 6, 7, 8, 9, 10, 15, 20, 30, 40 50, 60, 70, 80, 90, 100, or more cell types. In some forms, the samples can include samples from multiple cell lineages, such as 2, 3, 4, 5, 6, 7, 8, 9, 10, 15, 20, 30, 40 50, 60, 70 ,80, 90, 100, or more cell lineages. In some forms, the samples can include samples from multiple tissues, such as 2, 3, 4, 5, 6, 7, 8, 9, 10, 15, 20, 30, 40 50, 60, 70, 80, 90, 100, or more tissues. In some forms, the samples can include samples from multiple organs, such as 2, 3, 4, 5, 6, 7, 8, 9, 10, 15, 20, or more organs.

For training data sets, it is useful to use samples from a range of individuals, such as 2, 3, 4, 5, 6, 7, 8, 9, 10, 15, 20, 30, 40 50, 60, 70, 80 , 90, 100, or more individuals.

In some forms, one or more of the samples can include a heterogeneous mixture including a multiplicity of different cell types. In some forms, two or more cells of a same cell type can include two or more different cell regulatory states.

In some forms, one or more of the samples can include a peripheral blood sample from a subject. In some forms, the peripheral blood sample can include a multiplicity of leucocyte cells. In some forms, two or more leucocyte cells can include two or more different cell regulatory states.

In some forms, one or more of the samples can be derived from a biological sample at different time points. In some forms, one or more of the samples can be derived from a biological sample before and after treatment of the biological sample with an active agent. In some forms, the active agent can include a therapeutic agent, or a toxin.

In some forms, one or more of the samples can be derived from a biological sample before and after being depleted of all cells that include one or more cell subtype(s). In some forms, the depleted cells can include an abundant cell type in the first sample that are depleted from the second sample.

In some forms, the long reads of the long-read DNA methylation sequence data in the first omics data set can include about 3,000 to about 100,000 nucleotides, inclusive. In some forms, the single-cell-resolved reads of the single-cell-resolved DNA methylation sequence data in the second omics data set can include about 50 to about 600 nucleotides, inclusive.

In some forms, the first omics data set can include sequencing data with a depth of coverage of more than 300, preferably of more than 600, inclusive, for each nucleotide that is sequenced.

In some forms, the first and/or second omics data set(s) can include M5C modification data. In some forms, the M5C modification data can include a complete data set for all methylcytosines within each read in the first and/or second omics data set(s).

In some forms, the first and/or second omics data set(s) can include M6A modification data. In some forms, the M6A modification data can include a complete data set for all methyladenosines within each read in the first and/or second omics data set(s).

In some forms, the first and/or second omics data set(s) can include M5C modification data and sequences that include bases with M6A modifications. In some forms, the M5C modification data and sequences that include bases with M6A modifications can include a complete data set for all methylcytosines and all methyladenosines within each read. In some forms, the first and/or second omics data set(s) can further include 5-hydroxy-methylCytosine modifications.

In some forms, the first omics data set can be generated via OXFORD NANOPORE TECHNOLOGIES™ sequencing. In some forms, the OXFORD NANOPORE TECHNOLOGIES™ sequencing can be performed using a high-throughput, real-time DNA and RNA sequencing device, such as an OXFORD NANOPORE TECHNOLOGIES™ PROMETHION™ DNA sequencer. In other forms, the first omics data set can be generated via a Pacific Biosciences REVIO™ SPRQ™-Nx long read DNA sequencing platform, for example, to enable long read sequencing with accurate reporting of M5C, hydroxy-M5C and M6A base modifications using SPRQ™ chemistry. In some forms, the second omics data set can be generated by single-cell Methyl-seq.

In some forms, the assembly of long-read DNA methylation sequence data can involve de novo DNA sequence assembly of long reads in the first omics data set into the eHCs. In some forms, the eHCs can constitute full-length methylated DNA sequence of entire chromosomes, sometimes including the centromeres. In some forms, the assembly of long-read DNA methylation sequence data can be performed using software for haplotype-aware de novo assembly of diploid genomes from long reads. In some forms, the software can include PHASEBOOK software. In some forms, the mapping of single-cell-resolved DNA methylation sequence data can be performed using available alignment software such as MAFFT, Minimap2, or MUMMEr4.

In some forms, each eHG can correspond to the full chromosome complement of a cell regulatory state represented in the single-cell-resolved DNA methylation sequence data. In these forms, the single-cell-resolved DNA methylation sequence data constitutes cell-regulatory-state-resolved DNA methylation sequence data. In some forms, the full chromosome complement of each cell regulatory state can include repetitive DNA sequences that are differentially methylated in stressed or diseased cells. In some forms, the cell regulatory states can include one or more disease states.

In some forms, prior to production of the first and second omics data sets, the first and second samples or their source sample can be depleted of the most highly abundant cell subtypes. The resulting first and second sample(s) can be referred to as depleted first and second samples.

In some forms, one or more additional first and second omics data sets can be produced from one or more additional first and second sample derived from the same source as the first and second samples of a given pair of samples. In some forms, the additional first and second samples or their source sample can be depleted of the most highly abundant cell subtypes. The resulting first and second sample(s) can be referred to as depleted first and second samples.

In some forms, the “most abundant” cell type, subset, or cell regulatory state within a sample includes a cell type, subset, or cell regulatory state whose cells are present in the largest number relative to the cells of any other cell type, subset, or cell regulatory state(s) within the sample. For example, the most abundant cell type within a sample is that which includes the greatest number of individual cells of the same type. A single cell type, subset, or cell regulatory state is the most abundant when it is present as 100% of the total number of cells in a sample. More typically, a single cell type, subset, or cell regulatory state is the most abundant when its cells are present in an amount less than 100% of the total number of cells in a sample, but is present as a greater percentage of the total than the percentage of cells of any other single cell type, subset, or cell regulatory state in the sample. In some forms, the cells of the most abundant cell type, subset, or cell regulatory state in a sample are present in an amount that is 1, or 2, or 3 orders of magnitude greater than the cells of a less abundant cell type, subset, or cell regulatory state. Therefore, in some forms, the cells of a most abundant cell type, subset, or cell regulatory state are present in an amount that is between about 1000 times and about 1.1 times, inclusive, relative to the cells of a less abundant cell type, subset, or cell regulatory state within the same sample. For example, in some forms, the cells of a most abundant cell type, subset, or cell regulatory state are present in a relative amount that is at least 1000 times, or less than 1000 times the amount of cells of a less abundant cell type, subset, or cell regulatory state within the same sample, such as 900, 800, 700, 600, 500, 400, 300, 200, or 100 times, or less than 100 times the amount of cells of a less abundant cell type, subset, or cell regulatory state within the same sample. In some forms, a most abundant cell type, subset, or cell regulatory state is present in a relative amount that is 99, 90, 80, 70, 60, 50, 40, 30, 20, 10, 9, 8, 7, 6, 5, 4, 3, 2 or less than 2 times the amount of cells of a less abundant cell type, subset, or cell regulatory state within the same sample, such as 1.9, 1.8, 1.7, 1.6, 1.5, or less than 1.5 times the amount of cells of a less abundant cell type, subset, or cell regulatory state within the same sample.

In some forms, one or more of the most abundant cell type(s) is depleted from one or both of the omics datasets within a paired sample, for example, by chemical and/or physical isolation and separation from the sample. In some forms, one or more of the most abundant cell type, subset, or cell regulatory state(s) is depleted from both of the first and second omics datasets within a paired sample. In other forms, one or more of the most abundant cell type, subset, or cell regulatory state(s) is depleted from only the first or only the second omics datasets within a paired sample. Typically, for creation of a depleted sample, the top 1, 1-2, 1-3, 1-4, or 1-5 most abundant cell types, subsets, or cell regulatory states are depleted from the first, second or both omics datasets. Depletion is typically designed to increase the relative abundance and relative accuracy of sequences of the less-abundant cell types, subsets, or cell regulatory states as compared to the corresponding un-depleted samples. The depletion of one or more abundant cell types, subsets, or cell regulatory states from a sample to provide a depleted sample can include a complete depletion or incomplete depletion of the abundant cells from the sample. For example, in some forms, a depleted sample can include about 0% of a depleted cell type relative to a non-depleted sample. In other forms, a depleted sample includes less than 100% of the original amount of one or more abundant cell types, subsets, or cell regulatory states, such as from about 0.1% to about 99% of the original amount of the abundant cells relative to the non-depleted sample. Typically, a depleted sample includes a reduction in the amount of one or more abundant cell types relative to a non-depleted sample, such that the depleted cell type is no longer present in an amount greater than one or more other cell types in the sample. Therefore, in some forms, a depleted sample includes from about 0.1% up to 99% of a most abundant cell type relative to a non-depleted sample. In some forms, a depleted sample includes sequence data derived from about 1%, up to about 99%, inclusive, of the total number of cells within the corresponding non-depleted sample. In some forms, a single cell dataset includes data for a single cell type, subset, or cell regulatory state derived from about 50- to about 100 individual cells. In some forms, a depleted sample includes less than 10% of the number of cells of a most abundant cell type, subset, or cell regulatory state as compared with a corresponding non-depleted sample.

In some forms, long-read DNA methylation sequence data derived from one or more depleted first samples can result in a larger proportion of the eHICs being derived from cell types and/or cell lineages present at low frequency in undepleted samples as compared to when long-read DNA methylation sequence data derived from one or more undepleted first samples are assembled into eHCs. In some forms, single-cell-resolved DNA methylation sequence data derived from one or more depleted second samples can result in a larger proportion of the eHCs being derived from cell types and/or cell lineages present at low frequency in undepleted samples as compared to when single-cell-resolved DNA methylation sequence data derived from one or more undepleted second samples are assembled into eHCs.

In some forms, the methods can further involve formulating the eHCs as a training set for a machine learning architecture. In some forms, the architecture can include a foundation model and a transformer. In some forms, the model and transformer each can include encoding systems for training based on the training set of eHCs and corresponding second omics data sets. In some forms, the model and transformer can learn the sequence relationships among all of the eHGs.

In some forms, the methods can further involve generating, using the machine learning architecture, one or more further eHGs from one or more further omics data set(s) that each include further long-read DNA methylation sequence data derived from one or more further sample(s). In some forms, the machine learning architecture can generate a plurality of eHGs each corresponding to the full chromosome complement of respective different single cells represented in the further long-read DNA methylation sequence data of the further omics data set(s). In some forms, each eHG can represent a cell regulatory state for a single cell in the further sample(s).

Also disclosed are methods involving analyzing a test omics data set derived from a test sample using a deep learning (DL) model and assigning cell regulatory states to cells in the test sample without a need for analyzing single-cell omics data from the test sample. In some forms, the method can further involve displaying the assigned cell regulatory states on a graphical user interface in real-time. In some forms, the assigned cell regulatory states can be displayed on a graphical user interface within 1, 2, 3, 4, 5, 10, 15, 20, or no more than 30 minutes after receiving the eHC-assembled test omics data set.

In some forms, the DL model can be trained on one or more first omics data set(s) from one or more first sample(s) and one or more second omics data set(s) from one or more second sample(s). In some forms, each first sample in the one or more first sample(s) can constitute a pair of samples with one of the second sample(s) in the one or more second sample(s). In some forms, the first and second samples of a given pair of samples can be derived from the same source and include overlapping cell types and/or cell lineages. In some forms, the test sample can be derived from the same type of source as the source of at least one of the pairs of first and second samples.

DL models are generally the best suited to convert bulk genomics sequencing data sets to virtual single-cell data sets. An important improvement inherent in the disclosed DL model is that it enables the generation of 46-chromosome whole-genome data sets. These data are of significant utility because, for the first time, it is possible to assemble cell-type-specific, or lineage-specific, full chromosomal complements from bulk deep DNA sequencing data. However, other models can be used, such as diffusion models (Schuette et al., 2025).

In some forms, training the DL model can include the DL model learning relationships among the first and second omics data sets. In some forms, during training the DL model can learn and associate subsets of data within the first and second omics data sets as belonging to a single cell. In some forms, during training the DL model can learn and associate subsets of data within the first and second omics data sets as belonging to an individual cell regulatory state.

In some forms, the methods can further involve fine-tuning the DL model with one or more additional first and second omics data sets relevant for one or more of the cell regulatory states of the source of the samples. In some forms, the additional first and second omics data sets can be produced from one or more additional first and second sample derived from the same source as the first and second samples of a given pair of samples. In some forms, the additional first and second samples or their source sample can be depleted of the most highly abundant cell subtypes. The resulting additional first and second samples can be referred to as depleted first and second samples.

In some forms, the DL model can be a foundation model. In some forms, the DL model can be a language model. In some forms, the DL model can include a Hyena architecture or a transformer architecture. In some forms, the DL model can include a HyenaDNA architecture or a StripedHyena architecture. In some forms, the DL model can include a StripedHyena architecture.

In some forms, the DL model can tokenize data at molecular scale resolution. In some forms, the DL model can tokenize data at single-nucleotide resolution.

In some forms, the test omics data set from the test sample can include long-read DNA methylation sequencing data of the test sample. In some forms, the first omics data set from the first sample(s) can include long-read DNA methylation sequencing data of the first sample(s). In some forms, the second omics data set from the second sample(s) can include single-cell-resolved DNA methylation sequencing data of the second sample(s).

In some forms, the test omics data set from the test sample can include DNA having between 3,000 and 100,000 nucleotides. In some forms, the first omics data set from the first sample(s) can include DNA having between 3,000 and 100,000 nucleotides.

In some forms, the second omics data set derived from the second sample(s) can include single-cell-resolved DNA methylation sequence data. In some forms, the second omics data set from the second sample(s) can include DNA having between 100 and 600 nucleotides.

In some forms, assigning cell regulatory states to cells in the test sample without a need for analyzing single-cell omics data from the test sample can involve querying the trained DL model with a test long-read DNA methylation sequence data set derived from the test sample and assigning epi-Haplotype Chromosomes (eHCs) to cell regulatory states. In some forms, the eHCs can be single-chromosome-resolved DNA methylation sequence data generated from assembly of the test long-read DNA methylation sequence data followed by a query that is processed by the trained DL model.

In some forms, one or more of the omics data sets can include a modality selected from the group consisting of single-cell transcription (RNA-seq) profiles, single-cell transposase chromatin accessibility profiles (ATAC-seq) profiles, single-cell M6A-MTase chromatin accessibility (MTase-seq) profiles, single-cell genome-wide DNA methylation (Methyl-seq) profiles, single-cell chromosome conformation capture (Hi-C-seq) profiles, bulk transcription profiles generated from whole-tissue RNA (bulk-RNA-seq) profiles, and bulk Deep long-Read DNA methylation profiles.

In some forms, one or more of the omics data sets can include a modality where the modality can include DNA sequences with genomic coordinates. In some forms, the DNA sequence data can further include M5C modification data. In some forms, the DNA sequence data can further include sequences including bases with M6A modifications. In some forms, the DNA sequence data can further include M5C modification data and sequences including bases with M6A modifications. In some forms, the DNA sequence data can further include 5-hydroxy-methylCytosine modifications.

In some forms, one or more of the samples can include a heterogeneous mixture including a multiplicity of different cell types. In some forms, two or more cells of a same cell type can include two or more different cell regulatory states.

In some forms, one or more of the samples can include a peripheral blood sample from a subject. In some forms, the peripheral blood sample can include a multiplicity of leucocyte cells. In some forms, two or more leucocyte cells can include two or more different cell regulatory states.

In some forms, one or more of the samples can be derived from a biological sample at different time points. In some forms, one or more of the samples can be derived from a biological sample before and after treatment of the biological sample with an active agent. In some forms, the active agent can include a therapeutic agent, or a toxin.

In some forms, one or more of the samples can be derived from a biological sample before and after being depleted of all cells that include one or more cell subtype(s). In some forms, the depleted cells can include an abundant cell type in the first sample that are depleted from the second sample.

Also disclosed are non-transitory computer-readable mediums. In some forms, the computer-readable mediums can include executed instructions stored thereon. In some forms, the executed instructions can be executed by a processor to perform a method that can involve using a deep learning (DL) model to assign, in real time, cell regulatory states to cells in one or more test sample(s) without a need for analyzing single-cell omics data from the test sample(s).

In some forms, the DL model can be trained on at least one or more first omics data set(s) from one or more first sample(s) and one or more second omics data set(s) from one or more second sample(s). In some forms, each first sample in the one or more first sample(s) can constitute a pair of samples with one of the second sample(s) in the one or more second sample(s). In some forms, the first and second samples of a given pair of samples can be derived from the same source and can include overlapping cell types and/or cell lineages. In some forms, the test sample can be derived from the same type of source as the source of at least one of the pairs of first and second samples. In some forms, the DL model can be operably linked to a graphical user interface. In some forms, one or more of the samples can include a heterogenous population of cells.

### C. Systems and Methods for Multi-Omics Analyses of DNA Samples

Systems and methods that combine large-scale omics diverse datasets and pretrained transformers for developing foundation models have been established. Generative foundation models employing combinations of single-cell data and bulk tissue data for multi-omics analyses are provided.

A new class of omics information derived from analysis of bulk DNA samples is just starting to become available, consisting of single-molecule, long-read DNA methylation data. This remarkable technological advance has been most thoroughly documented by Kolmogorov et al., 2023. This research group has shown that it is possible to achieve state-of-the-art genomic structural variation calling performance using long OXFORD NANOPORE TECHNOLOGIES™ long reads. They complemented the sequencing protocols with a computational pipeline called Napu (OXFORD NANOPORE TECHNOLOGIES™ Analysis Pipeline) that produces haplotype-resolved de novo assemblies, along with phased small variants, SVs and methylation calls. Their sequencing and informatics pipelines are available as open source.

When raw electronic signals generated when DNA or RNA passes through a nanopore, which are then converted into a nucleotide sequence through a process called “basecalling”, such as implemented in OXFORD NANOPORE TECHNOLOGIES™ data, has been obtained utilizing sequencing long reads over 12 kb in length and if, additionally, the depth of genomic coverage is at least 300× (over 300 reads per haploid genome), it becomes possible to generate de-novo DNA sequence assemblies including the full length of entire chromosomes, with the genomic assembly sometimes extending across the centromere. If the original sample of cells is heterogeneous and includes many distinct cell types that exist in different cell regulatory states, as would be the case for white cells in a peripheral blood sample, one expects that the whole-chromosome de-novo sequenced assemblies obtained from a bulk DNA sample will resolve into many classes of epi-Haplotypes. Each class of epi-Haplotype assembly, denominated “eHap”, represents the chromosomal pattern of sequence modifications (based on a 5-base or 6-base alphabet) characteristic of a specific cell regulatory state. When an eHap assembly includes the entire sequence of a chromosome it is in some forms denominated as an eHC. Note that each of the eHap DNA assemblies readily obtainable at this stage of analysis corresponds to a single chromosome, not to a full chromosomal complement of 22 pairs of autosomes plus 2 sex chromosomes. While single cells contain a full complement of 22 pairs of autosomes plus 2 sex chromosomes, the process of bulk DNA sequencing results in scrambling of individual chromosomes, with loss of information as to which chromosomes and, most importantly, which coordinated “chromatin states” existed together as a full chromosome set within any given single cell in the heterogeneous sample.

In some forms, the methods provide full sets of chromosome complements (e.g., 46 chromosomes in each set) corresponding to specific cell regulatory states for individual cells within a complex tissue sample. Typically, the methods combine single-cell-resolved methylated DNA sequence data derived from single cells of a biological sample with long-read methylated DNA sequence data derived by deep-sequencing of a pool of cells from the same biological sample, to generate a multiplicity of distinct, full sets of chromosome complements associated with single cells from a multiplicity of cells derived from a complex tissue. In some forms, the methods accurately map and determine the DNA methylation states of essentially all repetitive elements from the DNA sequences within the pool of cells.

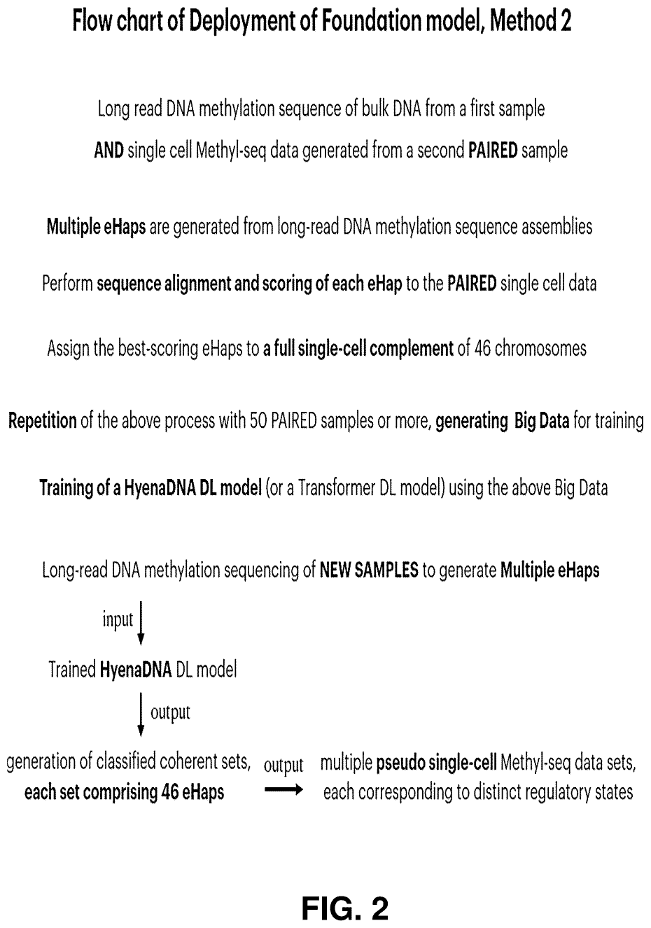

In other forms, the methods provide “pseudo” single-cell methylated DNA sequence analyses based on long-read methylated DNA sequence data derived by deep-sequencing of a pool of cells. In some forms, the methods utilize DNA sequences of full sets of chromosomes from each of the single cells, using a sufficiently large and wide mixture of cells, for example, such as those derived from a whole animal, whole organ, or whole tissue, to train and/or refine a transformer to produce a foundation model of the sequences and omics data of the assembled chromosome sets, and then produce a “generated” omics data set from a second or further mixture of cells derived from a second or further sample (see FIG. 2). Typically, the methods further include querying the trained foundation model with the generated omics data set to provide pseudo-single-cell-chromosome omics data for the generated data set. In some forms, the methods provide sets of coherent eHCs, each set representing a different full set of chromosomes from a different pseudo-single cell (e.g., an eHG) represented in the generated omics data set. In some forms, the methods provide a range of different eHGs (each eHG representing a different full set of chromosomes from a different pseudo-single cell), where a set of such eHGs represents the range of cell types in the mixture of cells in the second or further sample. In some forms, the methods provide output data that also reflect pseudo-single-cell omics data for the different pseudo-single cells represented in the second or further sample.

The disclosed approaches, which are compatible with a wide range of omics modalities, are high-throughput, scalable to large sample sizes, and massively increase the efficacy because they are uniquely amenable to automation. They also reduce the time and cost to implement as compared to other existing methods. The methods can be carried out using a range of omics data sets derived from a variety of existing techniques/existing databases, having different base modification sequencing information for a range of different genomic DNA fragments without the need for customized sequencing data sets. Therefore, the methods are compatible with standard universal DNA sequencing library preparation protocols, including deep sequencing, bisulfite sequencing, and methyl-seq data. Preferably, the methods produce data that is of equal quality or of better quality than data obtained using other genotyping/haplotyping by sequencing methods that do not employ machine learning techniques.

Methods for using the described systems to inform disease states in a subject are also provided. Typically, when the methods identify one or more cell regulatory states associated with a disease or disorder in a sample derived from a subject, the methods identify the subject as having a disease or disorder, and frequently enable a precise classification of a disease subtype. The methods are compatible with a broad range of complex biological samples, such as clinical samples, blood samples, tumor biopsy samples, etc. Samples obtained from a subject can be selected to provide haplotyping data and/or diagnostic information for a specific disease, disorder or cellular state.

### 1. Systems and Methods for Chromosome-Level De Novo Genome Assembly Including Base Modifications

Systems and methods of providing full sets of chromosome complements corresponding to specific cell regulatory states for individual cells within a complex tissue sample are provided. In some forms, the cells are human cells, and the methods provide a full set of methylated DNA sequences for each of 46 chromosomes associated with each cell type/cell regulatory state within a multiplicity of cell types/cell regulatory states derived from a biological sample from a subject.

In some forms, the methods involve the assembly of long-read DNA methylation sequences obtained from deep sequencing into an entire sequence of methylated DNA corresponding to each of the chromosomes (“eHCs”) from one or more cells within a mixture of cells from a biological sample, followed by mapping of single-cell DNA methylation data to each of the eHCs to provide a complete set of 46 eHCs associated with each cell regulatory state within the mixture of cells. When an eHap assembly includes the entire sequence of a chromosome or a chromosome-identifying sub-chromosomal eHap it can be denominated as an eHC.

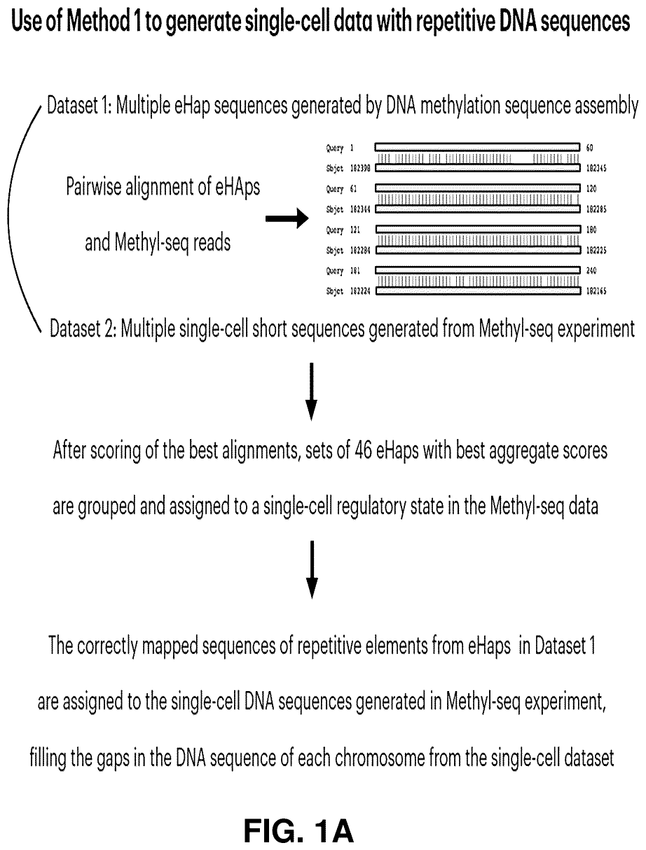

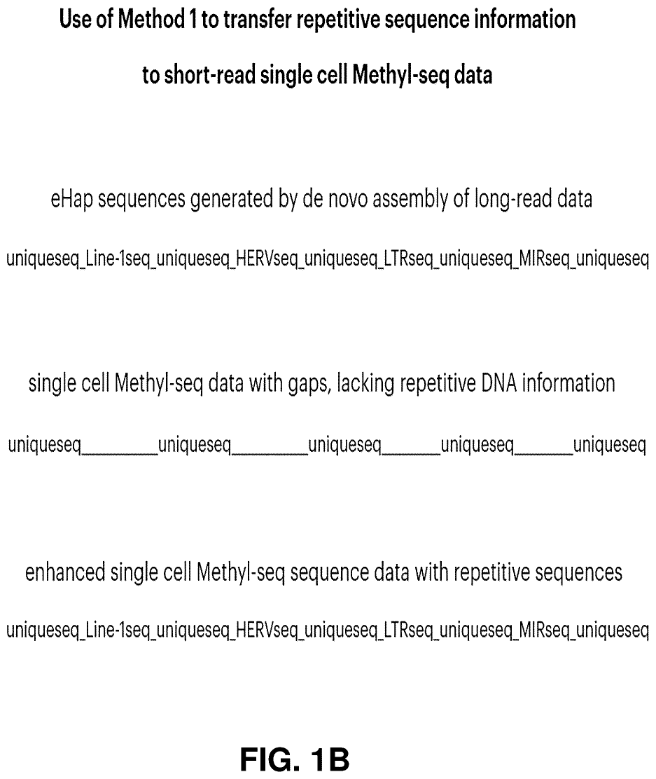

In some forms, the methods are implemented on a computer, for example, using a computer processor operably linked to means for providing computer software, and means for visualizing and entering controls and/or output. Therefore, in some forms, DNA sequences are provided in the form of a computer readable format, such as bit stream data. In some forms, the methods store DNA data including different de-novo assemblies that represent the epigenetic variability of each individual chromosome within a matrix. In some forms, each different chromosome assembly represents a single epi-Haplotype Chromosome (eHC). Typically, the methods extract and re-assign the information residing at highly abundant genomic loci corresponding to repetitive elements. Highly abundant genomic loci corresponding to repetitive elements are not mapped to specific genome positions in the single-cell Methyl-seq data set due to the typically short DNA sequencing reads. The resulting data sets include whole genome grouping assemblies including full coherent sets of 46 eHCs. Typically, each set of 46 eHCs is assignable to an individual cell regulatory state defined by Methyl-seq data. The methods augment the information content of the corresponding Methyl-seq data by the addition of locus-specific DNA methylation states for all repetitive DNA elements in the genome (see FIGS. 1A-1B). Notably, this information is missing in the original single-cell Methyl-seq data sets.

In some forms, the methods include one or more steps of AI-based transformation of the acquired data. Typically, AI-based transformation includes assignment of DNA methylation eHC sequence assemblies, and reassignment of DNA methylation at highly abundant genomic loci corresponding to repetitive elements.

The resulting data sets include whole genome grouping assemblies of full coherent sets of 46 eHCs, each set of 46 eHCs specifically assignable to a specific cell regulatory state defined by Methyl-seq data. Most importantly, each corresponding single-cell Methyl-seq data set is now dramatically augmented in its information content by the inclusion of locus-specific methylation sequences for all repetitive DNA elements in the genome (see FIGS. 1A-1B).

In some forms, the methods include one or more of the following steps (i)-(vii), including:
(i) providing a first omics data set and a second omics data set derived from a first complex biological sample;
(ii) assembling long-read methylated DNA sequences from the first omics data set obtained from the first sample to provide a methylated DNA sequence of each eHC within the first sample(s);
(iii) mapping single-cell-resolved read methylated DNA data sequences from the second data set to eHCs assembled in (ii), to associate each eHC assembled from the first data set with a single cell represented in the second data set;
(iv) assignment of individual eHC assemblies to individual cell regulatory states, wherein the assignment assigns a full chromosomal complement of 46 eHC sequence assemblies as belonging to the entire diploid complement epigenome of each specific cell regulatory state derived from the first complex biological sample;
(v) optionally repeating steps (i)-(iii), or (i-iv), using first and second paired data sets from biological samples depleted of the most highly abundant cell subtypes in the biological sample;
(vi) formulating the eHCs determined in (iv) and/or (v) as a training set for a machine learning architecture including a Foundation model and a transformer,
wherein the model and transformer learn the sequence relationships among all sets of 46 eHC genome complements assigned to corresponding single-cell data in the training set; and
(vii) providing a third or further omics data set(s) including long-read DNA methylation sequencing data for a multiplicity of cells derived from a third or further biological sample, and classifying eHCs derived from the third or further omics data set(s) as the output of the machine learning architecture,
optionally wherein the classified eHCs include complete sets of 46 eHCs, each representing an individual cell regulatory state for a single cell within the third or further biological sample.

Each of the above steps (i)-(viii) is described in more detail, below.

i. Providing a First Omics Data Set and Second Omics Data Set

The methods include one or more steps of providing a first omics data set and a second omics data set derived from a first complex biological sample.

Typically, the first and second omics data sets include one or more data sets derived from one or more paired samples. Each paired sample typically includes a first sample formed of a multiplicity of different cells and/or cell types from a complex biological sample, and a second sample, preferably with identical or substantially identical composition, derived from the same complex biological sample.

In certain forms, when the first omics data set represents the genomic nucleic acid content of a multiplicity of cells having a variety of cell types and cell regulatory states, for example, a blood sample from a subject, the second omics data set represents the genomic nucleic acid content of distinct classes or lineages of single cells from the same blood sample from the same subject. Therefore, in some forms, the second omics data set provides the genomic nucleic acid content of each of several defined subsets of the first omics dataset.

a. First (Long Read) Omics Data Set

Typically, the methods include providing a “first” data set including methylated DNA sequencing data derived from deep sequencing of nucleic acids derived from a complex biological sample, for example, including a variety of different cell types and cell regulatory states, present with different abundances within the sample.

Typically the first omics data set includes “long-read” DNA methylation sequences derived from deep sequencing of a biological sample. Typically the first data set is paired with a “second” data set derived from the same biological sample. Typically the first data set includes longer nucleic acid sequences than those within the “second” data set.

In exemplary forms, the first omics data set is made up of deep, long-read DNA methylation data including sequences in the size range of from about 3,000 to about 100,000 nucleotides, inclusive, preferably of about 4,000 to about 80,000 nucleotides, inclusive. In exemplary forms, the first omics data set, includes sequences having a depth of coverage of more than 300, preferably of more than 600, inclusive, for each nucleotide that is sequenced 300 times, up to about 600 times, or more than 800 times. In some forms, the first omics data set includes the presence of 5-methylcytosines within the long reads. In exemplary forms, the first omics data set is generated using an instrument with the characteristics of the OXFORD NANOPORE TECHNOLOGIES™ PROMETHION™ instruments. In other exemplary forms, the first omics data set is generated using an instrument with the characteristics of a Pacific Biosciences REVIO™ SPRQ™-Nx long read DNA sequencing platform.

b. Second (Single-Cell-Resolved) Omics Data Set

Typically, the methods include providing a “second” data set including methylated DNA sequencing data derived from bisulfite sequencing of nucleic acids derived from a single cell derived from the same complex biological sample as the first omics data set. In some forms, methylated DNA sequencing data can be generated using enzymatic methods for identification of 5-methylcytosine bases, which represent an alternative to bisulfite sequencing. In some forms, the generation of enzymatic sequencing data can utilize methods available as commercial kits from Biomodal, Cambridge CB101XL, UK, or alternatively Bimodal, Carlsbad, CA 92001, USA.

Therefore, in some forms, the second and first omics data sets are derived from a paired sample. A paired sample, such as a paired biological sample, includes a first subset of a complex biological sample that includes a mixture of cells or mixture of nucleic acids obtained from a mixture of cells and a second subset of the same sample. In some forms, the second data set includes nucleic acids that are labelled and/or tagged as being derived from a specific source, such as a single cell. In some forms, the second omics data set includes the sequences of labelled or tagged nucleic acids derived from one cell or cell type. In some forms, the second omics data set includes the sequences of genomic nucleic acids derived from a multiplicity of different cells, organized within the data set such that the cellular origin of each nucleic acid sequence is known. For example, in some forms, the second omics dataset provides a multiplicity of nucleic acid sequences derived from each of the 46 chromosomes of a human cell.

Typically, the second data set is paired with a first data set derived from the same biological sample. Typically, the second data set includes shorter nucleic acid sequences than those within the first data set. In some forms, the single-cell-resolved read sequences in the second omics data set include a multiplicity of nucleic acid sequences of about 50 to about 600 nucleotides, inclusive, typically about 150 to about 500 nucleotides, inclusive.

In some forms, the second omics data set is a single-cell-resolved MethylSeq data set derived from many distinct classes of cells, where, for each class of cells, the data set provides unique MethylSeq information corresponding to a cell regulatory state. In these forms, the single-cell-resolved MethylSeq data set constitutes cell-regulatory-state-resolved MethylSeq data. Several different methods are available in the art for creating a Methyl-seq data set, for example, as described in Spix, et al., Nat Commun. 2025 Jul. 8; 16(1): 6273; and Iqbal and Zhou, Computational Methods for Single-cell DNA Methylome Analysis. Genomics Proteomics Bioinformatics. 2023 February; 21(1): 48-66, the contents of which are hereby incorporated by reference in their entirety. Methyl-seq data has well-defined genomic coordinates, with the exception of those Methyl-seq measurements corresponding to repetitive DNA elements, which cannot be mapped unambiguously from single-cell-resolved reads.

Sequence reads (e.g., short reads) included in the disclosed second data sets can be grouped into single-cell-resolved reads or can be grouped into a set of reads that all belong to the same cell using any suitable technique, such as cell barcoding methods. The most advanced barcoding workflow in use to generate single-cell data is combinatorial indexing by tagmentation and additional ligation of DNA tags. This is achieved by using a transposase that cleaves DNA almost at random, and at the same time adds a sequence tag, a process called tagmentation. Cell nuclei are tagged in large groups within microtiter wells, using 384 different indexing tags. The large groups are split into smaller groups and then tagmentated again with different tags. More tags may be added during the PCR amplification steps. The addition of tags to the DNA fragments originating in each individual cell is combinatorial in nature, such that the final reads obtained by sequencing can be indexed and assigned to a single cell of origin based on DNA sequences that include thousands of different combinations of indexing tags (see, e.g., Tu et al. 2022, FIG. 1 and entire paper). Single-cell-resolved reads can be grouped into a set of reads that all belong to the same cell regulatory state using the disclosed methods that use cell-regulatory-state-relevant methylation sequence data to identify single-cell-resolved reads into sets that have similar or identical methylation patterns.

ii. Assembling Long Methylated DNA Sequences in the First Omics Data Set

Typically, the methods include assembling the long DNA methylation reads from the first omics data sets within the first sample. The assembly process generates a methylated DNA sequence of each class of complete chromosome present within the first sample, which is represented as an epi-Haplotype Chromosome (eHC). Since a complex sample contains a multiplicity of cell types, the assembly process generates a distinct epihaplotype sequence (eHap) for each cell type, as well as for each of several cell regulatory states that a single cell type may display. As an example, macrophage cell types in blood may display several cell regulatory states, such as activated vs. non-activated macrophage. The methods record each assembled eHC within a first matrix.

Typically, the methods align overlapping long-read DNA sequences from the first omics data set (derived from deep sequencing of a pool of cells/cell types) to provide a multiplicity of linearly contiguous methylated DNA sequences corresponding to a multiplicity of entire chromosomes. Therefore, assembly includes de-novo DNA sequence assembly of a multiplicity of chromosomes from a multiplicity of sequence fragments. Typically, each assembly includes assembling a full-length methylated DNA sequence of an entire chromosome, including genomic assembly sometimes extending across the centromere of the chromosome.

In some forms, the step of assembly creates a database including the complete nucleotide sequences of entire chromosomes derived from the pool of cells, for example, where each cell from the pool of cells contributes 46 separate chromosomes/eHCs. In some forms, assembly utilizes software for haplotype-aware de novo assembly of diploid genomes from long reads, such as the PHASEBOOK software.

Assembly is based on nucleic acid and methylation sequence overlaps within the multiplicity of DNA sequences. Therefore, assembly cannot by itself determine the origin of each eHC within the pool of eHCs.

In an exemplary form, the methods assemble sequences of long DNA methylation reads, including Epihaplotype-resolved de novo sequence assembly of long DNA reads obtained from each sample using available software tools such as Phasebook (Luo, et al., Genome Biol. 2021 vol 22(1): 299). Since the tissue samples being analyzed are complex and contain many cell types, the resulting whole-chromosome DNA sequence de-novo assemblies will include a large number of different eHCs.

Different sub-chromosomal eHaps can be determined to belong to the same chromosome by, for example, (a) removing DNA methylation information from the methylated DNA sequence of a sub-chromosomal eHap, thus presenting the sequence of the sub-chromosomal eHap using the 4-base alphabet, (b) choosing a few query subsegments in the 4-base sequence of the sub-chromosomal eHap, each more than, for example, 20,000 bases long and less than, for example, 25,000 bases long, and (c) feeding those subsegment sequences to BLAT (internet site genome. ucsc. edu/cgi-bin/hgBlat) or a similar alignment tool. BLAT will align all subsegments to the reference Human genome, and the output will identify which chromosome aligns perfectly with all the subsegments. The largest repetitive DNA elements in the genome are about 12,000 bases long, therefore query subsegments longer than this allows BLAT to identify chromosomes without risk of confusion due to sequence repeats.

iii. Mapping DNA Sequences From the Second Omics Data Set to eHCs

The methods include mapping the single-cell-resolved read DNA data from the second data set to the eHCs assembled in (ii). The single-cell-resolved read DNA data from the second data set represent different classes of cells, such as different subtypes of cells or cells in different cell regulatory states. The methods systematically align the methylated DNA sequences from each class of the single-cell-resolved read DNA data with each and every one of the eHCs assembled from the first data set. Therefore, the mapping step identifies each class of methylated nucleic acid sequence in the second data set as being associated with the best fitting eHC methylation sequence represented in the first data set, as determined by the aggregate sequence alignment scores.

Typically, mapping includes scoring an alignment between each available individual eHC chromosome assembly assembled in (ii) against the second data set containing the DNA methylation information of individual single cells from paired samples. In some forms, mapping in further includes loading the eHC data from (ii) in a second matrix, wherein the second matrix includes the same structure and dimensions as the first matrix, and wherein sequencing data from both the first and second data sets are aligned and mapped to the same genome.

In some forms, the mapping utilizes the alignment software MAFFT. In some forms, the mapping uses the alignment software Minimap2. In some forms, the mapping uses the alignment software MUMMEr4. In some forms, the mapping includes alignments of a pool of DNA sequences from the first data set with DNA sequences from the second data set, wherein each of the DNA sequences within the pool of first DNA sequences includes a methylated DNA sequence of a chromosome (eHC), and where the DNA sequences from the second data set include methylated DNA sequences from different classes of single cells, including about 50 to about 600 nucleotides inclusive, preferably from about 50 to about 600 nucleotides inclusive, and where the second DNA sequence may include defined genomic coordinates.

While each single cell in a heterogeneous sample contains a full complement of 22 pairs of autosomes plus 2 sex chromosomes, the process of DNA sequencing from a bulk sample preparation results in scrambling of individual chromosome fragments from different cells, with loss of information as to which chromosomes and, most importantly, which specific “chromatin states” existed together as a functional whole-epigenome within any given single cell.

In some forms, the mapping includes alignment of methylated DNA nucleotides in the DNA sequences of the eHCs assembled in (ii) with the sequences in the second omics data set, for example, wherein the alignment includes one or more steps of
(a) aligning each methylated DNA sequence from the second omics data set with each of the eHCs assembled in (ii); and
(b) scoring the sequence alignment.

In an exemplary form, the initial set of data from paired samples includes at least two whole-genome omics analysis modalities. Preferred modalities are deep, long-read DNA methylation sequencing, and additionally, from the same tissue sample, data generated by single-cell Methyl-seq. The objective of the scoring is to identify the best matches of sequence alignment in eHC assemblies that accurately map to specific cellular states as defined by the metrics of each single cell in the second omics data set (e.g., Methyl-seq data set). The methods typically require a first omics data set prepared from deep sequencing with a coverage depth greater than or equal to about 300×, in order to generate eHCs derived from low-abundance cells in the sample.

In some forms, the methods load the eHC data in an array with exactly the same structure and dimensions as an array used to load Methyl-seq data, taking advantage of the fact that both methylation data sets are mapped to the same (e.g., human) genome sequence. The Methyl-seq data is typically derived from single-cell-resolved reads and will include DNA fragments with a length of about 150 to about 500 bases. Since repetitive sequences and their methylation patterns are mapped accurately in the long-read eHC assembly, while mapping is not possible for the single-cell-resolved Methyl-seq data, the repetitive component of the genomic data sets will be loaded but will not be used in the scoring of methylation matches across the genome.

iv. Assignment of eHC Assemblies to Individual Cell Regulatory States

The methods assign individual eHCs to an assembly of 46 chromosomes, based on the association of eHCs with sequences from the second omics data set. In some forms, the scores derived from the mapping step are used to assist the assignment.

The methods include one or more steps to assign each of the individual DNA methylation eHC sequence assemblies generated in step (ii) to a single-cell status, such as a cell regulatory state, based on the mapping in (iii). Typically, the assignment of an eHC sequence assembly is determined by the corresponding Methyl-seq analysis of the sample.

For example, in some forms, the methods assign a full chromosomal complement of 46 eHC sequence assemblies (from a large set of different eHC assemblies) as belonging to the entire diploid complement epigenome of each specific cell regulatory state. The methods, at this step of analysis, do not include highly abundant genomic loci corresponding to repetitive elements in the assignment step. Highly abundant genomic loci corresponding to repetitive elements are not mapped to specific genome positions in the Methyl-seq data set due to the short DNA sequencing reads and therefore are not utilized during the assignment process.

Typically, the assignment includes allocating each of the sets of 46 eHC sequence assemblies as belonging to the entire diploid complement epigenome of each specific cell regulatory state, as determined by sequence and methylation patterns.

The methods assign a full chromosomal complement of 46 eHC sequence assemblies from a large set of eHC assemblies (generated in step (ii)) as belonging to the entire diploid complement epigenome of a specific cell regulatory state, with this process repeated for each and every instance of a distinct cell regulatory state represented in the data set of the paired second sample.

By selecting eHC matches yielding the best aggregate sequence alignment scores for single-cell-resolved reads, the methods assign a full set of 46 eHCs to each cell regulatory state identified from the single-cell-resolved data of the second sample. The methods repeat this process for each instance of a single-cell methylation profile in the data set.

Maternal and paternal eHC pairs will have the property of belonging to eHC assemblies with moderate differences in DNA methylation. Genomic loci accounting for differences in the maternal and paternal eHC pairs typically include, for example, the small subset of imprinted genes with different allele-specific methylation patterns and/or levels. Additionally, maternal and paternal eHC pairs should have almost exactly the same number of sequencing reads assignable to the sequence assembly contigs. In those cases where the number of single cells analyzed by Methyl-seq is considerably larger (say, 16,000) than the sequencing depth utilized for the long-read DNA methylation analysis (say, 600×), there will likely exist a larger number of cell regulatory states relative to the total number of different eHC sequence assemblies. In this instance, the methods will assign a subset of different cell regulatory states to the same chromosomal complement of eHC sequences. This numerical discrepancy underscores the need for a balanced experimental design, achieved by using a larger long-read sequencing depth (i.e. 1,000×) that more closely matches the number of possible different cell regulatory states.

In some forms, the methods assign eHCs based on the scoring in step (iii), for example, by
(a) selecting eHC matches yielding the best aggregate sequence alignment scores for each single-cell-resolved DNA sequences corresponding to a cell regulatory state, and
(b) assigning a full set of 46 eHCs to each cell regulatory state.

In some forms, assignment in step (iv) includes
(a) extracting information from each highly abundant genomic locus corresponding to a repetitive element, and
(b) re-assigning information from highly abundant genomic locus corresponding to repetitive elements,
wherein the assigning includes addition of locus-specific DNA methylation states for all repetitive DNA elements in the genome.

Different sub-chromosomal eHaps can be determined to belong to the same chromosome from the same cell by, for example, first joining available sub-chromosomal eHaps to form the longest possible eHap that is tagged as corresponding to one of the 46 chromosomes. In most cases, if assembly spanning the centromere is not achieved, the sub-chromosomal eHaps will be successfully joined to form two complete chromosome arm (pter, qter) assemblies for each chromosome. The next step is to perform pairwise alignments to single-cell data (as described above and elsewhere herein), and to score the alignments to generate eHGs (as described above and elsewhere herein).

v. Repeating Steps (i)-(iii) or (i-iv) Using Depleted Biological Samples

The methods include one or more steps of optionally repeating (i)-(iii) or (i-iv) using biological samples depleted of the most highly abundant cell subtypes in the biological sample. In some forms, the methods include providing sequencing data from a depleted biological sample that includes
(a) a first depleted data set including long-read sequencing data including DNA derived from a multiplicity of cells from the depleted biological sample; and
(b) a second depleted data set including single-cell-resolved DNA sequences derived from a single cell from the same depleted biological sample.

In some forms, repeating the mapping in (iii) one or more times using the depleted biological sample, wherein the methods repeat the analysis in steps (i-iii) using the depleted first sample and depleted second sample to refine and improve the assignment in step (iv)

In some forms, the methods repeat one or more of steps (i), (ii) (iii) and (iv) to generate omics data sets and map eHC assemblies to individual cell regulatory states as defined by Methyl-seq analysis using biological samples depleted of those cells that include one of the most highly abundant cell subtypes in the biological sample. In some forms, repetition of the analysis in (i), (ii) and (iii) using depleted samples serves to refine and improve the assignment in step (iv).

Typically, the assembly of sequences in the first depleted data set in step (ii) provides a larger proportion of eHCs derived from cell types present at low frequency as compared to assembly of the corresponding first sample. Therefore, the mapping of sequences from the second depleted data set to eHCs in step (iii) aligns a larger proportion of single-cell-resolved methylated DNA sequences derived from cell types present at low frequency as compared to mapping of the corresponding second sample.

vi. Formulating EHCs as a Training Set for Machine Learning

In some forms, the methods include formulating the eHCs as a training set for a machine learning architecture. Typically, the architecture includes a foundation model and a transformer, for example, whereby the model and transformer include encoding systems for training based on the training set of eHCs and paired second omics data sets, and whereby the model and transformer learn the sequence relationships among all sets of 46 eHC genome complements assigned to corresponding single-cell data in the training set.

In an exemplary form, the methods extract and re-assign the information residing at highly abundant genomic loci corresponding to repetitive elements. These elements are not mapped to specific genome positions in the Methyl-seq data set due to the short DNA sequencing reads, and therefore they are not utilized during the assignment process in step (iv). However, in the deep long-read DNA methylation data sets obtained from bulk samples, repetitive elements are indeed mapped accurately at over one million unique positions in the human genome.

Therefore, in some forms, the methods generate a new matrix where each repetitive DNA sequence present in coherent eHC data sets is assigned to each corresponding empty matrix position in the corresponding single-cell Methyl-seq data set.

vii. Providing a Third or Further Omics Data Set(s)

In some forms, the methods include providing a third or further omics data set(s) including long-read DNA methylation sequencing data for a multiplicity of cells derived from a second or further biological sample and generating full chromosome complements of 46 eHCs for the third or further omics data set(s) through deployment of the machine learning architecture. In some forms, the machine learning architecture provides a multiplicity of complete sets of 46 eHCs, wherein each complete set of 46 eHCs represents an individual cell regulatory state for a single cell within the second or further biological sample.

### 2. Systems and Methods for Generating Single-Cell DNA Methylation Information From Bulk DNA Sequencing Data Sets

Systems and methods of providing individual cell regulatory state information from a complex biological sample are also provided.

In some forms, the methods include analyzing a paired sample from the complex tissue sample to generate a training data set, analyzing one or more further sample(s) from the complex tissue sample using the training data set, and then assigning DNA methylation eHC sequence assemblies of each of the complete chromosome sequences to one or more cellular statuses for the one or more further sample(s). Typically, the methods assign DNA methylation eHC sequence assemblies for a further sample in the absence of single-cell analysis of the further sample.

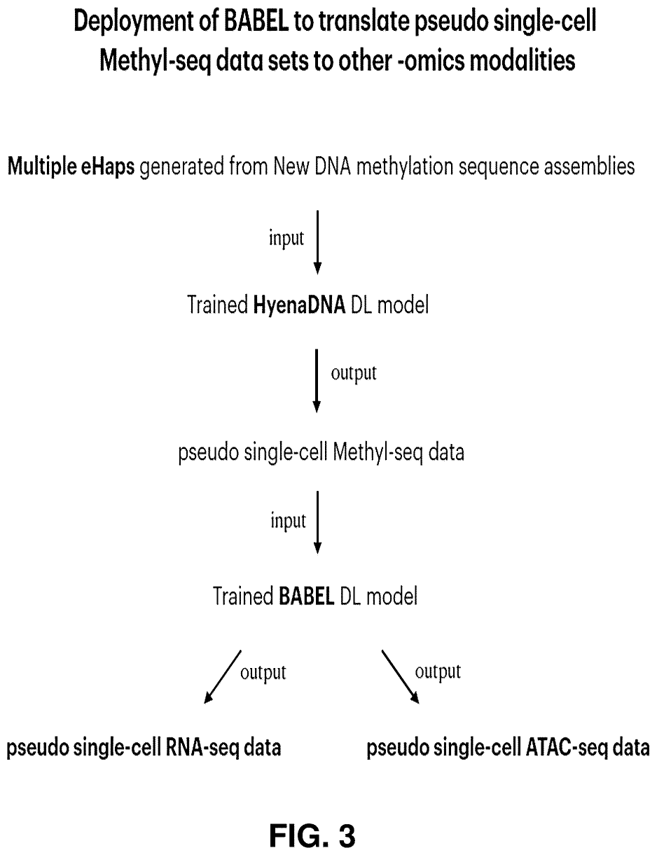

Analyzing cell heterogeneity and understanding individual components of a heterogeneous cell population is desired for clinical applications, such as immune diagnostics. In some forms, the methods determine a molecular profile of chromatin-based cell regulatory states of one or more different cells within a heterogeneous biological sample, such as a clinical sample, without single-cell analysis of the individual cells within the sample. This can be achieved by deployment of the BABEL cross-modality translation architecture to further process the data generated by the disclosed Hyena DNA Deep Learning Foundation model, which is illustrated in FIG. 3.

In some forms, the sample is a stored or archived sample, such as frozen tissues or frozen blood, or a cellular component or lysate thereof. In some forms, the sample is not amenable and/or suitable for single-cell analysis.

The methods include one or more steps of generating a training data set, for example, utilizing an initial analysis of a moderate-sized, representative subset of paired samples, including two or more omics analysis modalities. In some forms, a first modality includes DNA methylation sequencing data derived from a “bulk” or complex biological sample, and a second modality, performed on replicate samples of the same tissue, includes single-cell analysis data. The methods utilize the bimodal analysis of paired samples as initial input for a foundation model based on training of a transformer analogous to language transformers. The methods employ a foundation model with an architecture based on a dual trained and fine-tuned transformer according to the input modality omics data sets, to analyze further data set(s) as input, whereby the further data set(s) derived from a further complex sample.

Importantly, the machine learning methods included in the disclosed HyenaDNA or the transformer architecture provide the capability for analysis of the further data set(s) derived from a complex sample without the need for corresponding further single-cell data.

In some forms, a further data set includes an omics modality derived from a further complex biological sample that is derived from the same or different subject, tissue, and/or cell type than that from which the initial input data. Therefore, in some forms, the methods generate pseudo-single-cell resolution analysis of cell regulatory states for cells from a previously un-analyzed bulk sample. In some forms, the methods use the training model to query a multiplicity of further data sets, for example, derived from a multiplicity of complex samples. In some forms, the further data sets are larger than the initial input data set including, for example, nucleic acid and methylation data associated with a larger number samples or a larger number of cells. In some forms, the cells are human cells, and the methods provide a full set of methylated DNA sequences for each of 46 chromosomes associated with each cell type/cell regulatory state within a multiplicity of cell types/cell regulatory states derived from a further complex biological sample from a subject.

In some forms, the methods include one or more of the following steps (i)-(vii), including:
(i) analyzing one or more paired samples from the complex tissue to train or train and refine a DL foundation model including, but not limited to, a StripedHyena architecture, a HyenaDNA architecture, or transformer architecture that has been trained to learn all the relationships among the DNA methylation sequences of assembled chromosome sets and the omics data generated by single cell analysis within the paired sample;
(ii) analyzing one or more further sample(s) using the foundation model produced in (i); and
(iii) assignment of DNA methylation eHC sequence assemblies of each of the complete chromosome sequences to one or more cellular statuses for the one or more further sample(s).

Each of the above steps (i)-(viii) is described in more detail, below.

i. Analyzing a Paired Sample From the Complex Tissue Sample to Train a Transformer and Produce a Foundation Model

The methods include one or more steps to provide individual cell regulatory state information from a complex biological sample by analyzing a paired sample from the complex tissue sample to generate a training data set. Typically, the paired sample includes a first sample and a second sample from the complex tissue sample. In some forms, the first sample includes a first omics modality, and the second sample includes a second omics modality. In some forms, the first modality includes DNA methylation sequencing of a heterogeneous mixture of cells from the first samples, and the second modality includes single-cell analysis, performed on a replicate sample of the first sample.

In some forms, the methods include one or more steps of providing a first omics data set and a second omics data set derived from a first complex biological sample. Typically, the first and second omics data sets include one or more data sets derived from one or more paired samples. Each paired sample typically includes a first sample formed of a multiplicity of different cells and/or cell types from a complex biological sample, and a second sample, preferably with identical or substantially identical composition, derived from the same complex biological sample.

In certain forms, when the first omics data set represents the genomic nucleic acid content of a multiplicity of cells having a variety of cell types and cell regulatory states, for example, a blood sample from a subject, the second omics data set represents the genomic nucleic acid content of distinct classes of single cells from the same blood sample from the same subject. Therefore, in some forms, the second omics data set provides the genomic nucleic acid content of each of several defined subsets of the first omics dataset.

Typically, the methods include assembling the long DNA methylation reads from the first omics data sets within the first sample. The assembling generates a methylated DNA sequence of each chromosome present within the first sample, which is represented as an eHC. The methods record each assembled eHC within a first matrix.

Typically, the methods align overlapping long-read DNA sequences from the first omics data set (derived from deep sequencing of a pool of cells/cell types) to provide a multiplicity of linearly contiguous methylated DNA sequences corresponding to a multiplicity of entire chromosomes. Therefore, assembly includes de-novo DNA sequence assembly of a multiplicity of chromosomes from a multiplicity of sequence fragments.

Typically, each assembly includes assembling a full-length methylated DNA sequence of an entire chromosome, including, in preferred forms, a genomic assembly that sometimes extends across the centromere of the chromosome.

Typically, the methods assign individual eHCs to an assembly of 46 complete chromosomes, based on the association of eHCs with sequences from the second omics data set. In some forms, the scores derived from the pairwise sequence alignment step are used to assist the assignment. The methods include one or more steps to assign each of the individual DNA methylation eHC sequence assemblies.

The methods further include loading the eHC data in an array with exactly the same structure and dimensions as the array used to load the second omics (i.e. Methyl-seq) data, taking advantage of the fact that both methylation data sets are mapped to the same human genome sequence. The second omics (i.e. Methyl-seq) data is derived from single-cell-resolved reads and includes DNA fragments with a length of 150 to 500 bases. The second omics (i.e. Methyl-seq) data has well-defined genomic coordinates, with the exception of those measurements corresponding to repetitive DNA elements, which cannot be mapped unambiguously from single-cell-resolved reads. Since repetitive sequences and their methylation levels are mapped accurately in the long-read eHC assembly, while mapping is not possible for the single-cell-resolved read second omics (i.e. Methyl-seq) data, the repetitive component of the genomic data sets will be loaded but will not be used in the scoring of methylation matches across the genome.

The methods further include aligning and scoring the sequence matches of the DNA sequence (using a 5- or 6-base alphabet) of each individual eHC assembly against second omics (i.e. Methyl-seq) sequences that are generated for each distinct type of cell regulatory state, limiting the scoring to unique-sequence DNA from the second omics (i.e. Methyl-seq) data. Typically, the methods for pairwise sequence alignment employ a DNA multiple sequence alignment computer program, such as MAFFT, Minimap2, or MUMMEr4. The pairwise sequence alignment process will typically include a large matrix of DNA sequence alignments of two DNA sequences with a length in the range of 150 to 500 bases. By selecting those eHC matches yielding the best aggregate sequence alignment scores for a single cell the methods assign a full set of 46 eHCs to each specific cell regulatory state identified by single-cell data. This process is repeated for each instance of a single-cell methylation profile in the second omics data set. Maternal and paternal eHC pairs will have the property of belonging to eHC assemblies with moderate differences in DNA methylation. Note that genomic loci accounting for differences in the maternal and paternal eHC pairs will include, for example, the small subset of imprinted genes with different allele-specific methylation patterns and/or levels. Additionally, maternal and paternal eHC pairs should have almost exactly the same number of sequencing reads assignable to the sequence assembly contigs. Where the number of single cells analyzed by second omics (i.e. Methyl-seq) data is considerably larger (say, 16,000) than the sequencing depth utilized for the long-read DNA methylation analysis (say, 600×), there will most likely exist a larger number of cell regulatory states relative to the total number of different eHC sequence assemblies. When this happens, the methods may perform suboptimally by assigning a subset of different cell regulatory states to the same chromosomal complement of eHC sequences. Preferably, the first omics data set includes a larger long-read sequencing depth of coverage (i.e. 1,000×) that more closely matches the number of possible different cell regulatory states, thereby avoiding suboptimal assignments.

The methods further include one or more steps to refine eHC assignments to cell regulatory states. In some forms, the methods repeat the analysis using biological samples depleted of those cells that include the most highly abundant cell subtype in the sample. For example, in human peripheral blood the most abundant type of white blood cells are neutrophils, which can be readily removed from samples of white cells using commercial kits. Depletion of a highly abundant cell type sharply and linearly alters the relative frequencies of ALL remaining cell subtypes. Improved accuracy in the analysis of depleted samples is due to the following: 1) the presence in the long-read DNA methylation analysis of bulk DNA of a much larger number of DNA sequencing reads derived from cell types present at low frequency, and; 2) in the single-cell analysis of depleted samples there will be a much larger representation of single-cell data generated for those cells present at relatively low frequency.

Typically, the methods process the data obtained from the depleted samples exactly as indicated above for the first and second omics data, to assign DNA methylation eHC assemblies to each instance of an individual cell regulatory state. The methods typically align/score the sequence (using a 5- or 6-base alphabet) of each individual eHC chromosome assembly against the second omics (i.e. Methyl-seq) data available for each single cell, limiting the scoring to unique-sequence DNA from the second omics (i.e. Methyl-seq) data. The methods select the matches with the best scores to assign a full set of 46 eHCs to each specific cell regulatory state identified by the single-cell second omics (i.e. Methyl-seq) data.

Typically, the methods involve training or training and refining a StripedHyena architecture, a HyenaDNA architecture, or a transformer architecture to develop and deploy a foundation model. In the preceding section(s) the assignment of whole-chromosome DNA methylation eHC assemblies to corresponding cell regulatory states defined by paired-sample single-cell data was described. This process generates complete sets of 22 pairs of eHCs plus 2 sex chromosome eHCs that contain the entire epigenome DNA sequence of each single cell that exists in a particular cell regulatory state. Each set of eHCs containing 22 pairs of autosomes plus 2 sex chromosomes is said to be “coherent” because it represents a collection of “chromatin states” that function together harmoniously to orchestrate a specific regulatory logic within a single living cell. An eHC is a distinct digital descriptor of the ensemble of the distinct “chromatin states” present in a chromosome. Since an eHC contains a linear DNA sequence of letters, it can be easily parsed into tokens, and the tokens can be encoded for language information processing. In addition to the DNA sequence token, which could contain 25 bases, a separate metadata track can be used to store genomic coordinates for each sequence token. Likewise, the Methyl-seq data contains short DNA sequences with specific genomic coordinates, and the sequences can also be parsed as tokens with a metadata track for storing coordinates. The long-read eHC data contains additional information elements relating to the structure of the “contigs” that were built during the process of sequence assembly that generated each eHC. A “contig” is set of DNA segments or sequences that overlap in a way that provides a contiguous representation of a genomic region built by sequence assembly. For any given eHC assembly the “count” is the number of DNA sequencing reads that participate in a contig assembly at specific windows of DNA sequence characterized by the highest difference in nucleotide sequence compared to all other eHC assemblies. In other words, the “count” is a property of a small subset of DNA sequence reads that participated in the de novo assembly of each eHC and, importantly, represent regions that define the identity and uniqueness of an eHC chromosomal DNA methylation pattern, as they do not exist in other eHCs. Thus, each eHC is associated with its own characteristic “count,” which can be encoded as a metadata vector in the data set. Preferably, the “count” for those eHCs belonging to a single coherent, whole-genome set of 46 eHCs are very similar in value, reflecting the relative abundance of the corresponding cells that exist in a given specific cell regulatory state. To be useful for training, the compendium of sets of coherent eHCs can be generated from the analysis of at least 50 tissue (or blood) samples, and can contain many hundreds or even thousands of whole genome, single-cell chromosomal complements with different methylation profiles. One thus proceeds to construct a machine learning system that is trainable based on the sequence information encoded in the previously generated long-read DNA methylation eHC data sets and the corresponding paired sample's single-cell Methyl-seq data sets. Since methylated DNA sequences are structurally similar to sentences in language, the existing deep learning HyenaDNA (Nguyen et al., 2023), StripedHyena (Nguyen et al., 2024), or transformer (Vaswani et al., 2017, Devlin et al., 2019, Szatata et al., 2024) tool sets utilized for DNA language or natural language processing can be adapted to this task. Detailed descripts of a HyenaDNA architecture, a StripedHyena architecture, and a transformer architecture are described in Nguyen et al., 2023; Nguyen et al., 2024; and Vaswani et al., 2017, Devlin et al., 2019, and Szatata et al., 2024, respectively, the contents of which are herein incorporated by reference.

In the case of alternative cell regulatory states, the logical relationships of methylated DNA sequence tokens among 46 members of a coherent set of chromosomes contain base modification patterns characteristic of active and inactive gene sets, as well as patterns of chromatin accessibility and the inter-related patterns of DNA methylation that occur in all 46 members of a full chromosome set, in coordination, within a specific cell regulatory context. The architecture of a DL model(s) described herein is trained to recognize many thousands of distinctive tokens of DNA sequence information that represent unique digital imprints present in every specific coherent set of 46 eHCs that are key components of the paired data sets used for training. For instance, the HyenaDNA architecture can be an attention-free architecture based on implicit convolutions (Nguyen et al., 2023); the StripedHyena architecture can be based on a hybrid of layers of data-controlled convolutional layers interleaved with layers of multihead attention equipped with rotary position embeddings (Nguyen et al., 2024); and the transformer model can be based on an attention block (Szatata et al., 2024). The architecture thus will acquire, after training with the available paired eHC and single-cell data sets, the information needed to perform the future task of grouping a multiplicity of new eHCs into optimal coherent sets of 46 eHCs each.

By encoding the entire set of eHCs containing 22 pairs of autosomes plus 2 sex chromosomes corresponding to one specific cell regulatory state of a single cell, and then systematically performing this encoding process for all the known cell regulatory states in a heterogeneous tissue experiment, big data sets required for deep learning of the base sequence information relationships among eHCs, are generated, which belong to distinct sets of 46 full chromosomal complements that contain a large compendium of cell regulatory states. The data set used for training consists of labeled sets of 46 eHC sequences. The number of labels corresponds to the number of distinct cell regulatory states. Preferably, the data set is from a eukaryotic organism.

a. Hyenadna Architecture and Stripedhyena Architecture

The assignment of whole-chromosome DNA methylation eHC assemblies to corresponding cell regulatory states defined by paired-sample single-cell data was described above. This process generates complete sets of 22 pairs of eHCs plus 2 sex chromosome eHCs that contain the entire epigenome of each single-cell that exists in a particular cell regulatory state. Each set of eHCs including 22 pairs of autosomes plus 2 sex chromosomes is said to be “coherent” because it represents a collection of “chromatin states” that function together harmoniously to orchestrate a specific regulatory logic within a single living cell. An eHC is a distinct digital descriptor of the ensemble of the distinct “chromatin states” present in a chromosome. Each set of coherent 46 eHCs that contain a distinct cell regulatory state, specified by scoring of alignments with paired-sample single-cell data, is defined by specific DNA sequences that have distinguishing features based on a 5 or 6 letter DNA methylation alphabet.

The long-read eHC data contains additional information elements relating to the “count” of DNA sequence reads that participated in the de novo assembly of each eHC. Thus, each eHC is associated with its own characteristic “count”, which can be encoded as a metadata vector in the data set. Preferably, the “count” for those eHCs belonging to a single coherent, whole-genome set of 46 eHCs are very similar in value, reflecting the relative abundance of the corresponding cells that exist in a given specific cell regulatory state. To be useful for training, the compendium of sets of coherent eHCs can be generated from the analysis of at least 50 tissue (or blood) samples, and can contain many hundreds, possibly thousands of whole genome, single-cell chromosomal complements with different methylation profiles. While 50 paired tissue samples are sufficient for the training task described herein, 100 paired samples are preferred, 200 paired samples are more preferred, and 400 or more paired samples are most preferred. One thus proceeds to construct a machine learning foundation model that is trainable based on the sequence information encoded in the previously generated long-read DNA methylation eHC data sets.

Recently Hyena (Poli et al, 2023), a large language model based on implicit convolutions, was shown to match machine learning attention mechanisms in quality while allowing longer context lengths and lower time complexity. Leveraging Hyena's long-range capabilities, Nguyen et al. (2023) described HyenaDNA, a new genomic foundation model that can be pretrained on very long human genome sequences with context lengths of up to 1 million tokens at the single nucleotide-level-an up to 500× increase over previous dense attention-based models. HyenaDNA scales sub-quadratically in sequence length (training up to 160× faster than a Transformer), uses single nucleotide tokens, and has full global context at each layer. Transformers, while successful in natural language processing, struggle with long DNA sequences at single-nucleotide resolution. Their computational cost increases quadratically with input length, leading to limitations in context length and forcing the use of tokens that aggregate nucleotides. By contrast, HyenaDNA, a hybrid architecture combining attention mechanisms with data-controlled convolutional operators, excels at efficiently processing long sequences at single-nucleotide resolution.

StripedHyena uses layers of data-controlled convolutional operators (Hyena layers) intertwined with multi-head attention layers equipped with rotary position embeddings. Hyena layers effectively filter noise common in DNA sequences and aggregate individual nucleotides into meaningful motifs. The attention layers can take into account the relative positions of nucleotides in the sequence. Rotary position embeddings are a type of positional encoding that can be used in attention layers to help the model learn the order of the input sequence. They work by encoding the position of each element in the sequence as a rotation in a high-dimensional space. This allows the attention mechanism to take into account the relative positions of elements when computing the attention weights. The hybrid design, blending Hyena and Transformer components, improves scaling performance in DNA sequence modelling. Notably, on a species classification task, HyenaDNA was able to effectively solve the challenge and achieve high accuracy by increasing the context length to 1M bases without down-sampling.

The evaluation of DNA methylation sequence information relationships existing among a large collection of eHC sequences containing, for example, a possible 27 full chromosome complement sets (that is, 27 different cell regulatory states), each set composed of 46 chromosomes, can be analogous to the problem of classifying different DNA sequences from 27 different species of mammals. For instance, the 27 species of mammals could correspond to 27 different cell regulatory states, and their species-specific genomic DNA sequences could correspond to long range DNA methylation sequences of different eHCs. A trained HyenaDNA Foundation model using long DNA sequences from each of the 27 different mammal species, each long DNA sequence having a label corresponding to the species name as well as the chromosome number where the training set DNA came from, can be used. When challenged with a new set of long, unlabeled DNA sequences, the trained HyenaDNA Foundation model would be able to identify and add the correct species label and chromosome label to each of the unknown DNA sequences. This long-range species classification task is analogous to the task of classifying new eHC DNA sequences as belonging to a specific cell regulatory state, except that the task described herein is less complicated, because the total number of chromosomes in humans is 46, while among 27 different species of mammals the chromosome number will vary.

The above analogy can be useful for understanding the nature of the tasks to be accomplished by the new machine learning Foundation model described herein, and also serves to make clear what is meant by “coherent” in the description of distinct sets of 46 eHCs, each “coherent” set of 46 assignable to different instances of alternative cell regulatory states. In the case of alternative cell regulatory states, the interrelationships among methylated DNA sequences in 46 members of a full coherent set of chromosomes include base modification patterns characteristic of active and inactive gene sets, as well as patterns of chromatin accessibility and the inter-related patterns of DNA methylation that occur in all 46 members of a full chromosome set, in coordination, within a specific cell regulatory context. By encoding the entire set of eHCs including 22 pairs of autosomes plus 2 sex chromosomes corresponding to one specific cell regulatory state of a single cell, and then systematically performing this encoding process for all the known cell regulatory states in a heterogeneous tissue experiment, we can generate big data sets required for deep learning of the base sequence information relationships among eHCs that belong to distinct sets of 46 full chromosomal complements that include a large compendium of cell regulatory states.

Preferably, all eHCs that belong to different sets display distinct DNA methylation patterns that can be discovered by HyenaDNA since the task is mathematically the same as the species classification task.

Performing a training task for eHIC classification using HyenaDNA proceeds as follows:
(i) a large collection of eHCs is prepared as previously indicated, whereby each eHC, (previously generated by DNA sequence assembly) is assigned to a distinct cell regulatory state (regstate). Each eHC has two associated scalar labels, a regstate label, and a human chromosome_number label. A third metadata label is the “count,” which is the numeric value of sequence oversampling (also known as depth) present in the DNA sequencing reads participating in the contigs that map at the most highly eHC-differentiating positions of the individual sequence assembly. The large collection of labeled eHCs is processed using the “Species Classification” downstream task in the published HyenaDNA implementation (available at the world wide website “github. com/HazyResearch/hyena-dna”).

After training, the HyenaDNA Foundation model can be deployed to identify and add the correct regstate label and chromosome_number label to each new test sequence in a large collection of eHC DNA sequences, generated from long-read DNA methylation sequencing experiments without paired sample information.

It was pointed out earlier that each eHC is associated with its own characteristic “count”, which can be encoded as a metadata vector in the data set. The “count” for those eHCs belonging to a single coherent set of 46 eHCs should be very similar in value, reflecting the relative abundance in tissue (or blood) of the cell types that exist in any specific cell regulatory state. Therefore, the “count” data elements can be fine-tuned to error-check whether or not each set of 46 coherent eHCs satisfies the requirement for roughly equal count values for the eHCs within a candidate set under evaluation.

When presented with large sets of DNA sequence assemblies containing entirely new, not paired eHC data, the trained HyenaDNA Foundation model will be able to calculate the probability that any new eHC belongs to the same full, coherent chromosomal complement of 46 eHCs, said set of 46 eHCs corresponding to a specific cell regulatory state. In this manner the trained HyenaDNA Foundation model utilizes its training-generated information relationships to generate a multiplicity of new whole genome grouping assemblies of eHaps and eHCs, each grouping assembly including a coherent set of 22 pairs of autosomes plus 2 sex chromosomes.

The StripedHyena architecture can be based on a hybrid of layers of data-controlled convolutional layers interleaved with layers of multi-head attention equipped with rotary position embeddings (Nguyen et al., 2024). The data-controlled convolutional layers can be hyena layers, such that the StripedHyena architecture includes features of an improved hybrid HyenaDNA and transformer design.

b. Transformer Architecture

After encoding the labeled eHC data, one generates the corresponding Key matrix, Query matrix and Value matrix elements, as is done in the construction of language transformers.

The Key matrix is used to represent the identity or uniqueness of each input sequence element. It stores a vector representation of each input token, capturing its semantic and syntactic properties. The Key matrix helps the model identify and match similar elements across different eHC sequences during attention calculations.

The Query Matrix represents the information that the model is seeking to retrieve from the input sequences. It stores a vector representation of the current query or context, which defines the focus of attention. Each individual Key embedding is compared to all Query embeddings, resulting in pairwise attention scores. The Query matrix guides the attention mechanism to concentrate on all relevant parts of the input eHC sequences. The Query Matrix operates within a context window, which allows the Query Matrix to integrate information from nearby tokens, in order to discover and learn associations between tokens. Notably, the context window may also include tokens of information present in different eHCs within the same coherent set of 46 eHCs. In this manner machine learning discovers DNA methylation sequence relationships among different eHCs within the same coherent set. This holds true for all other coherent sets of eHCs.

The Value Matrix contains the actual information that the model wants to extract from the input sequences based on the query. It stores a vector representation of each input token, capturing its semantic and syntactic properties of DNA methylation patterns. The Value matrix provides the content that is weighted and combined based on the attention scores to produce the output sequences that contain the most valuable information for the desired goal. In essence, the Key, Query, and Value matrices work together to perform attention calculations, which allow the model to weigh the importance of different input eHC sequence elements based on their relevance to the current query. This attention mechanism plays an important role for capturing long-range dependencies among members of a distinct set of coherent eHCs, as well as understanding the overall context of the input data including all eHCs (coherent as well as non-coherent eHCs).

Attention heads can be set up to facilitate tuning of weights assigned to the encoded information residing within the sets of coherent eHC elements. The complex digital “syntax” and “grammar” of DNA methylation tokens present in coherent sets of eHCs will contain patterns of information directly related to gene regulation and coordinated gene expression occurring in different chromosomes that together, within a single nucleus, collaborate in the generation of a specific cell regulatory state. As discussed earlier, each eHC is associated with its own characteristic “count”, which can be encoded as a metadata vector in the data set. The “count” for those eHCs belonging to a single coherent set of 46 eHCs should be very similar in value, reflecting the relative abundance in tissue (or blood) of the cell types that exist in any specific cell regulatory state. Therefore, the “count” data elements can be used to error-check whether or not each set of 46 coherent eHCs satisfies the requirement for equal count values for the eHCs within a candidate set under evaluation.

Through the attention mechanism, the machine learning system is able to learn information patterns within an eHC, say, chromosome 19, that distinguishes it from another eHC of chromosome 19 that belongs to a different coherent set. Like is the case for chromosome 19, all eHCs that belong to different sets to display distinct information patterns (based on different DNA methylation patterns as well as the previously mentioned “count” metadata) that are discovered and annotated through a well-designed attention mechanism analogous to those used in human language transformers.

To extract additional useful information patterns, a set of Attention heads can be set up to focus self-attention in the information residing at millions of genomic loci corresponding to repetitive elements. During training and/or fine-tuning, weights can be optimized for capturing the information residing in the methylated sequences of repetitive elements that are correlated across different chromosomes within a single coherent set of 22 pairs of eHCs corresponding to autosomes plus 2 sex chromosomes. The extent of significant correlations existing across different chromosomes belonging to a single set of 46 eHCs may be significant, since any cell-specific alterations in the machinery that maintains methylation levels at repetitive element loci will cast its repressive epigenetic effects simultaneously across thousands of elements in the genome of the cell. A powerful approach to extract information from variation in the methylation states of repetitive elements in the use of an entropy metric, capable of quantifying the extent of order or disorder in the DNA methylation sequence patterns of each subset of repetitive elements belonging to the same family (i.e. Line-1, HERV, SVA, MER, etc.), as represented within each coherent set of 46 eHCs. When all encoded repetitive element information becomes part of the transformer attention matrix elements, the transformer utilizes features of repetitive element information as part of an enriched mathematical description of each specific, “coherent” set of 46 eHCs.

In this manner the repertoire of tools in language machine learning are utilized to train and/or fine-tune a transformer that learns the totality of semantic (sequence) and entropic (order/disorder) relationships among members of each individual set of 46 coherent eHCs, whereby a multiplicity of such coherent sets of 46 eHCs contains a large universe of different cell regulatory states.

When presented with large sets of DNA sequence assemblies containing entirely new, not paired eHC data, the trained transformer calculates the probability that any two or more eHCs belong to the same coherent chromosomal complement of 46 eHCs. This is accomplished by utilizing encoded sequence tokens for all relevant genomic loci, including hundreds of thousands of repetitive DNA elements, coordinate information for each relevant locus, tuned weights for different encodings, as well as information relating to the encoded “count” vector described in the previous paragraph. In this manner the transformer utilizes its training-generated information relationships to generate a multiplicity of new whole genome grouping assemblies of eHCs, each grouping assembly including a coherent set of 22 pairs of autosomes plus 2 sex chromosomes.

After training and/or fine-tuning, the relevant architecture, including, but not limited to HyenaDNA, StripedHyena, or transformer can be utilized to discriminate between coherent and non-coherent members of entirely new data sets containing a multiplicity of eHCs generated by long-read DNA methylation sequencing of a larger set of biological samples. For the analysis of these new complex tissues, paired Methyl-seq data will not be generated. Epihaplotype-resolved de novo sequence assembly of the long DNA methylation reads obtained from each sample can be performed as indicated earlier. As before, the resulting de-novo eHC assemblies will contain a large number of different epi-Haplotype Chromosomes.

Analysis of each of the new complex tissue(s) generates a large multiplicity of different eHCs. The relevant architecture evaluates available eHC sequence relationships and predicts a multiplicity of correct whole genome grouping assemblies of 46 coherent eHCs for each new tissue sample. The decoding output of the trained DL model will be a multiplicity of sets of coherent eHCs, each containing a full complement of 22 pairs of autosomes plus 2 sex chromosomes. The output may contain hundreds (or even thousands) of different sets of 46 eHCs, depending on the cell type regulatory complexity of each biological sample, facilitated by a high level of oversampling (300×, 400×, 600×, 800×, or more) achieved by the DNA methylation sequencing reads used for analysis.

Following the generation of all possible and distinct full sets of whole genome grouping assemblies including coherent eHCs, the trained model will automatically decode and predict the “virtual” pseudo-single-cell Methyl-seq profile that corresponds to each set of 46 coherent eHCs. Each of the states is called a “virtual” state because it is generated entirely by computation, and for the same reason the predicted single-cell data are called “pseudo-single-cell”.

ii. Extension of the Disclosed Methods to Include the use of Long-Read eHC Data in Combination With Two or Three Sets of Single-Cell Omics Data

The HyenaDNA Foundation model facilitates, in the context of this new DL model, for the deep data training that recognizes, via Dense convolutions, Element-wise gates, and Long implicit convolutions, hundreds of thousands of distinctive single-nucleotide tokens of DNA sequence information that represent distinct digital imprints present in every specific coherent set of 46 eHCs and also present in the paired data sets used for training. The HyenaDNA Foundation model, extended to work with 5-base or 6-base DNA methylation alphabets, thus acquires the information needed to perform the task of grouping a multiplicity of new eHCs into optimal coherent sets of 46 eHCs each. The most recent publication (Nguyen et al, 2024) on the development of machine learning Foundation models based on the Hyena architecture describes StripedHyena, a new architecture based on an improved Hyena hybrid design and scaled to a 1000× larger model size. The complex digital “syntax” and “grammar” of DNA methylation single-nucleotide tokens present in coherent sets of eHCs contain patterns of information directly related to gene regulation and coordinated gene expression occurring in different chromosomes that together, within a single nucleus, collaborate in the generation of a specific cell regulatory state.

It is also contemplated that the new DL models disclosed herein are applicable in settings that include the use of long-read eHC DNA methylation data in combination with two or three sets of single-cell omics data.

Those skilled in the art will recognize that the process described in steps above can be extended, using more powerful Foundation models, to include any number of additional sets of single-cell omics data, such as RNA-seq, ATAC-seq, of Hi-C-seq. The inclusion of additional information for paired samples, containing two or three sets instead of one set of single-cell omics data, can result in an improvement in the training process, an additional improvement in the subsequent fine-tuning process, as well as improvements in the selection and prediction process.

A preferred combination of two sets of single-cell omics data is Methyl-seq combined with single-cell Hi-C-seq (Ramani et al., 2017, 2020). Single-cell Hi-C-seq data is highly informative regarding the topology of chromatin loping in a cell, by generating a list of DNA ligation events that bring together distant DNA sequences. Attention heads or implicit convolutions can be added to the machine learning modules to effectively associate each of the methylation states defined by specific vectors in the embedded long-read DNA methylation data of each eHC with the corresponding set of chromatin looping contacts of a single-cell in a given cell regulatory state. In this manner, the machine learning architecture can associate each chromatin looping event with coordinated methylation changes observed at distant locations where chromatin contacts occur. The attention mechanism or implicit convolutions can facilitate capturing long-range dependencies among members of a unique set of coherent eHCs, resulting in the discovery of unsuspected topological relationships that may be present linking long-range DNA methylation patterns and Hi-C-seq data,

Another preferred combination of two sets of single-cell omics data is Methyl-seq combined with single-cell RNA-seq (see review by Gupta et. al, 2024). Since single-cell RNA-seq data is highly informative as to the exact cell regulatory state of a cell, attention heads or implicit convolutions can be added to the machine learning modules to effectively associate the different methylation states defined by specific vectors in the embedded data of each eHC with the observed regulatory states of each gene that maps near those positions in the eHC.

Yet another preferred approach involves a combination of three different single-cell omics data sets, namely Methyl-seq combined with single-cell ATAC-seq (CellSpace package for processing of DNA accessibility in chromatin, Tayyebi et al., 2024) and single-cell RNA-seq. Notably, the published CellSpace ATAC-seq work flow preserves precise DNA sequence information and genomic coordinates information associated with the DNA accessibility data, which greatly facilitates integration with Methyl-seq data.

A summary of the workflow for analyzing a new complex tissue(s) ensues. After training and/or fine-tuning using paired samples (by which whole-chromosome DNA methylation eHC assemblies have been associated with alternative cell regulatory states), it becomes possible to analyze a new (unpaired) complex tissue(s) without necessitating deployment of single-cell technologies, using only single-molecule, long-read DNA methylation sequencing, followed by de novo DNA sequence assembly. The process is designed to generate pseudo-single-cell resolution of different cell regulatory states present in a heterogeneous sample. The process includes one or more of the following steps:
(i) Generation of Deep, long-Read DNA methylation data (using a 5-base or 6-base alphabet) from a complex tissue(s), with a coverage equal to or greater than 300×;
(ii) De novo assembly of long, deep DNA methylation data reads, to create chromosome sequence assemblies (eHCs) that sometimes extend across the centromere of each chromosome;
(iii) Storage and encoding of each eHC sequence data in a matrix format;
(iv) Deployment of a trained deep learning HyenaDNA architecture, StripedHyena architecture, or Transformer architecture, capable of selecting groups of coherent eHCs (chromosome chromatin states) from large sets of bulk sequence data that contain a large multiplicity of different eHCs. Each of the selected groups of coherent eHCs, containing 22 pairs of autosomes plus 2 sex chromosomes, correspond to a distinct cell regulatory state; and/or
(v) Decoding of each full complement of eHCs containing 22 pairs of autosomes plus 2 sex chromosomes to generate the corresponding “virtual” pseudo-single-cell Methyl-seq profiles characteristic of specific cell regulatory states.

Preferably, the output of these models is displayed the results on a graphical user interface in real-time, such as within 1, 2, 3, 4, 5, 10, 15, 20, or no more than 30 minutes after receiving the eHC-assembled first omics data.

Also disclosed is a non-transitory computer-readable medium with executed instructions stored thereon executed by a processor to perform a method involving using a trained DL model described herein to assign, in real-time, single-cell regulation information to cells in a first complex tissue including a heterogenous population of cells without a need for analyzing single-cell omics data from the first complex tissue. The DL model has been trained on at least: a) a second omics data set from a second complex tissue including a second heterogenous population of cells, and (b) a second omics data set from replicate samples of single cells from the second complex tissue. Preferably, the DL model is operably linked to a graphical user interface.

iii. Alternative Machine Learning Tools That Can be Utilized to Build Foundation Models for Deep Learning According to the Methods Taught in This Disclosure

The practical implementation of the disclosed methods is not limited to the Foundation models described herein, and those skilled in the art will understand that as new Machine Learning tools are developed, these new tools can be readily adapted to the Deep learning tasks described herein. The published literature continues to be enriched with improved methods for implementation of Foundation models based on more efficient Deep learning architectures. Notable examples include Mamba (Gu & Dao, 2024), xLSTM (Beck et al, 2024) and Bio-xLSTM (Schmidinger et al, 2024), which are briefly described below.

Mamba incorporates a new class of selective state space model (SSM) that improves on prior architectures to achieve the modeling power of Transformers while scaling linearly in sequence length. The Mamba design implements a simplified selection mechanism that parameterizes the SSM parameters based on the input. This allows the model to filter out irrelevant information and remember relevant information indefinitely. The Mamba architecture combines the design of prior SSM architectures with the Multilayer Perceptron block of Transformers into a single block, leading to a simple and homogenous architecture design that incorporates Selective State Space Models. These Selective SSMs, and by extension the Mamba architecture, are fully recurrent models with key properties that make them suitable as the backbone of general foundation models operating on sequences. (i) High quality: selectivity brings strong performance on dense modalities such as language and genomics. (ii) Fast training and inference: computation and memory scales linearly in sequence length during training, and unrolling the model autoregressively during inference requires only constant time per step since it does not require a cache of previous elements. (iii) Long context: the quality and efficiency together yield performance improvements on real data up to sequence length 1 million.

The recently proposed Extended Long Short-term Memory (xLSTM, Beck et. al, 2024) is a powerful architecture for sequence modeling and a promising candidate for biological and chemical sequences. The xLSTM architecture introduces enhanced memory structures and exponential gates that boost its performance, particularly in natural language modeling. Despite these enhancements over traditional LSTM, xLSTM retains the efficiency of a recurrent neural network and can handle varying sequence lengths effectively, while maintaining expressivity and scalability. These features make xLSTM ideal for modeling DNA sequences, which are inherently long and for which long-range interactions between distant parts of the sequence have been observed.

Schmidinger et al. (2024) have introduced DNA-xLSTM, an architectural variant of Bio-xLSTM tailored for DNA sequences. DNA-xLSTM excels in preforming in-context learning (ICL), a capability of language models to learn and perform tasks by leveraging additional information provided as the contextual input without updating their parameters. This approach allows models to learn from analogy, drawing insights from patterns in the context to adapt their behavior. DNA-xLSTM leverages reverse-complement invariance, crucial for modeling DNA, and has shown competitive or superior performance compared to state-of-the-art models like DNA-Mamba on tasks such as promoter and splice site prediction.

The DNA-xLSTM architecture has enhanced sequence modeling capabilities, particularly for varying context lengths. There are three model configurations based on DNA-xLSTM: two sLSTM-based configurations trained with a context window of 1,024 tokens (DNA-xLSTM-500k and DNA-xLSTM-2M), and an mLSTM-based configuration trained with a context window of 32,768 tokens (DNA-xLSTM- 4M). In their publication, Schmidinger et al. (2024) demonstrated the potential of the Bio-xLSTM architecture as a prime candidate to model biological and chemical sequences. DNA-xLSTM showed strong performance in DNA sequence modeling, excelling in both masked and causal language tasks across different context sizes. These findings underscore the potential of DNA-xLSTM as a candidate for Foundation models in molecular biology and genomics, such as those described in this disclosure.

### 3. Preparation Of Nucleic Acid Samples

Any of the methods described herein can include one or more steps of preparing nucleic acid sequences from a biological sample, and/or preparing an omics data set including nucleic acid sequence data.

Methods for preparation of genomic nucleic acid sequences from biological samples, such as those including cells and/or tissues are known in the art. For example, methods for collecting various bodily or cellular samples and for extracting nucleic acids are well known in the art. In some embodiments, nucleic acid samples are obtained from cells, tissues, and/or bodily fluids containing nucleic acid. Exemplary source organisms for nucleic acid samples include animals, such as humans, and plants.

Exemplary biological samples include bodily sample including, but not limited to, tissue, organ(s), blood, lymph, urine, gynecological fluids, and biopsies. Bodily fluids can include blood, urine, saliva, or any other bodily secretion or derivative thereof. Blood can include whole blood, plasma, serum, or any derivative of blood. Typically, the sample includes cells, particularly eukaryotic cells from tissue(s) or from a biopsy. Samples can be obtained from a subject by a variety of techniques including, for example, by scraping, washing, or swabbing an area, by using a needle to aspirate bodily fluids, or by removing a tissue sample (i.e., biopsy).

If the nucleic acid sample is within cells, tissue or bodily fluids, preparation and purification of the nucleic acid from the sample can include lysis of cells, such as cells within blood. A nucleic acid sample, such as a nucleic acid sample obtained from a pool of different cells, or a nucleic acid sample obtained from a single cell derived from the pool of different cells can be provided as a lyophilized, dry powder, or in solution, such as an aqueous solution. Sequencing data, such as deep sequencing data, or methyl-seq data can be formulated into data sets associated with one or more omics modalities using techniques and procedures known in the art. In some forms, the entire diploid complement epigenome of each specific cell regulatory state includes repetitive DNA sequences that are differentially methylated in stressed or diseased cells

In some forms a pooled nucleic acid sample includes more than a single genomic DNA sample from a single cell. For example, a pooled nucleic acid sample can include genomic DNA from two different cells, or more than 2 cells, such as 100, 1,000, 10,000, 100,000 or more than 100,000 different cells. When nucleic acid samples contain genomic DNA from more than a single cell, the cells can be of the same type, or different types. In some forms, a pooled nucleic acid sample can include genomic DNA from two different cell types, or more than 2 cell types, such as 100, 1,000, 10,000, 100,000 or more than 100,000 different cell types. In other forms, a pooled nucleic acid sample can include genomic DNA from two different cell regulatory states of a single cell type, or more than 2 different cell regulatory states of a single cell type, such as 100, 1,000, 10,000, 100,000 or more than 100,000 different cell regulatory states of a cell type. Typically, a set of “paired” data sets from a sample include a first data set, including sequencing data derived from an entire pool of cells, including a multiplicity of different cell types, and/or a multiplicity of different cell regulatory states; and a second data set, including sequencing data derived from a single cell(s), optionally where each DNA fragment derived from a single cell includes a specific tag or marker, for example, that is associated with the cell or origin, where the single cell is derived from the same pool of cells and the same sample as the first data set.

i. Generation of Omics Data Sets From Paired Tissue Samples

In an exemplary method, data acquisition includes providing two or more omics data sets utilizing paired tissue samples. Typically, the tissue samples encompass cells and/or tissues or different types/subtypes and/or cell regulatory states and include both data from bulk cells/tissues, as well as data from single cells. For example, in some forms, a first data set includes long-read DNA methylation sequencing data, for example, obtained from sequencing of bulk sample(s). In some forms, the methods provide long-read DNA methylation sequencing data from a pre-existing data set, such as within a database. In other forms, the methods provide long-read DNA methylation sequencing data by de novo sequencing of a sample(s) to acquire data. In some forms, the first and/or second omics data set(s) include M5C modification data. Exemplary M5C modification data includes a complete data set for all 5-methylcytosines within each DNA sequence. In some forms, the first and/or second omics data set(s) includes sequences including bases with M6A modifications. In some forms, the first and/or second omics data set(s) further includes 5-hydroxy-methylCytosine modifications. In some forms, the first and/or second omics data set(s) includes M5C modification data and sequences including bases with M6A modifications.

a. Omics Analysis Modalities

Input omics data sets can be prepared according to any omics analysis modality where multimodal data is available as paired data from the same tissue sample.

Exemplary omics modality datasets include single-cell RNA-seq (transcription profiles), including a data structure of an array of expression scalar values with gene annotation; single-cell ATAC-seq (Transposase chromatin accessibility), including a data structure of an array of sequences with genomic coordinates; single-cell MTase-seq (M6A-MTase chromatin accessibility), including a data structure of an array of sequences including bases with M6A modifications; single-cell Methyl-seq (genome-wide DNA methylation) including a data structure of an array of sequences including bases with M5C modifications; single-cell Hi-C-seq (chromosome conformation capture) including a data structure of an array of sequences with genomic coordinates; bulk RNA-seq (transcription profiles generated from whole-tissue RNA) including a data structure of an array of scalar values with gene annotation; bulk Deep long-Read DNA methylation (whole tissue DNA, OXFORD NANOPORE TECHNOLOGIES™ PROMETHION™) including a data structure of DNA reads with genomic coordinates, and full sequence with M5C modifications.

b. Long-Read DNA Methylation Analysis Modalities

Long-read DNA methylation analysis from bulk samples can be prepared according to any approaches potentially amenable to de-novo assembly of long DNA sequencing reads, including, but not limited to Deep long-read DNA methylation to generate epi-haplotypes (5- or 6-base alphabet), utilizing whole-chromosome de novo assembly of Deep long reads that sometimes extends across the centromere of a chromosome; Deep long-read M6A-MTase accessibility data (5-base alphabet), which generates alternative sequence patterns of chromatin accessibility via whole-chromosome de novo assembly of Deep long reads that sometimes extends across the centromere of a chromosome; and Deep long-read DNA methylation combined with Deep long-Read M6A-MTase accessibility data (Deep SAM-seq, Leduque et al, 2024), e.g., obtained simultaneously (6-base alphabet), via de novo assembly of Deep SAM-seq reads that sometimes extends across the centromere of a chromosome.

### (1) Bisulfite Sequencing (Methyl-Seq)

In some forms, the methods employ bisulfite sequencing to determine DNA methylation. DNA methylation is an important epigenetic modification that reveals insights into gene regulation and typically refers to the methylation of the 5-carbon of the cytosine base. DNA methylation consolidates epigenetic states over cell replication and is extensively implicated in many biological activities. Many non-recurrent cellular alterations with phenotypic manifestation are inheritable over mitosis and coded into the epigenome.

Bisulfite sequencing methods rely on bisulfite conversion of DNA to detect unmethylated cytosines. Bisulfite conversion changes unmethylated cytosines to uracil during library preparation. Converted bases are identified (after PCR) as thymine in the sequencing data, and read counts are used to determine the percent methylated cytosines. Bisulfite conversion sequencing can be done with targeted methods such as amplicon methyl-seq or target enrichment, or with whole-genome bisulfite sequencing. Additionally, alternative chemistries like OxBS and TAB-Seq can be used with NGS for identification of hydroxy-methylation (5-hMc) in conjunction with methylation (5-mc) analysis.

Therefore, in some forms, sequencing of purified genomic DNA fragments includes any technique that utilizes bisulfite treatment of the DNA to convert cytosine residues to uracil, but leaves 5-methylcytosine (5mC) residues unaffected (Frommer, et al., Proc. Natl. Acad. Sci. USA, 89, p 1827-1831 (1992)). The region of interest is then amplified using modified primers (designed in regions devoid of CpG sites) and sequenced using conventional methods such as capillary-based electrophoresis or pyrosequencing (Hardenbol, et al., Nat. Biotechnol. 21, p. 673-678 (2003)).

Sequencing of genomic DNA subjected to sodium bisulfite conversion (MethylC-Seq) can enable single-base resolution, strand specific identification of methylated cytosines throughout the majority of the genome. Therefore, in some forms, bisulfite treatment introduces specific changes in the DNA sequence that depend on the methylation status of individual cytosine residues, yielding single-nucleotide resolution information about the methylation status of a segment of DNA. The presence of 5mC is inferred from comparing bisulfite-treated DNA sequences to an untreated reference.

Bisulfite sequencing poses challenges due to DNA damage, leading to the development of enzymatic-based methods, revolutionizing methylation sequencing for precise, reliable results. Enzymatic methylome analysis yields high-quality libraries with reduced DNA damage and enhanced CpG detection. Bisulfite sequencing typically detects both 5mC & 5hmC (EM-seq) or 5hmC alone (E5hmC-seq).

In some forms, the methods employ Methyl-seq analysis according to the methods set forth in Iqbal and Zhou, Computational Methods for Single-cell DNA Methylome Analysis. Genomics Proteomics Bioinformatics. 2023 Feb; 21(1): 48-66.

### (2) TET 1-Oxidation

In some forms, identification of Methyl-cytosine is enhanced by Tet1 oxidation chemistry. In standard bisulfite sequencing, 5mC cannot be distinguished from 5-hydroxymethylcytosine (5hmC) (Huang, et al., PLOS One, 5(1): e8888 (2010)), because both resist deamination by bisulfite treatment. Therefore, conversion of 5mC to 5caC through the activity of a Tet1 enzyme and 5hmC to 5fC through chemical conversion followed by bisulfite sequencing runs has also been exploited for the genome-wide sequencing of 5mC and 5hmC (see methods of Clark, et al., BMC Biol; 11: 4 (2013)).

TET proteins not only oxidize 5mC to 5hmC, but also further oxidize 5hmC to 5caC. 5caC exhibits similar behavior as unmodified cytosine after bisulfite treatment. This deamination difference between 5caC and 5mC/5hmC under standard bisulfite conditions inspired TAB-Seq. In this approach, a glucose moiety is introduced onto 5hmC using β-glucosyltransferase (BGT), generating β-glucosyl-5-hydroxymethylcytosine (5gmC) to protect 5hmC from further TET oxidation. After blocking of 5hmC, all 5mC is converted to 5caC by oxidation with excess of recombinant Tet1 protein. Bisulfite treatment of the resulting DNA then converts all C and 5caC (derived from 5mC) to uracil or 5caU, respectively, while the original 5hmC bases remain protected as 5gmC. Thus, subsequent sequencing will reveal 5hmC as C, providing an accurate assessment of abundance of this modification at each cytosine when combined with traditional bisulfite sequencing (Yu, et al., Cell, 149(6): 1368-80 (2012)). Further, Selective chemical oxidation of 5hmC to 5-formylcytosine (5fC) enables bisulfite conversion of 5fC to uracil (Booth, et al., Science, 336, p 934-7 (2012)).

Therefore, in some embodiments, DNA sequencing is performed using Tet-assisted Bisulfite Sequencing (TAB-SEQ).

ii. Depleted Samples

In some forms, the methods include preparing a depleted sample from a complex biological sample. Typically, a depleted sample lacks one or more of the components of the complex biological sample from which it is derived. In an exemplary form, a depleted sample lacks a specific cell type. For example, in some forms, a depleted sample lacks a most abundant cell type relative to a complex sample from which it is derived.

iii. Integration of Multimodal Data Across Biological Conditions or Depleted Samples

In a first example, samples are obtained at two or more time points, for example, before and after treatment with a pharmaceutical drug.

In a second example, samples are depleted of all cells that include e a highly abundant cell subtype. Typically, this forms of depletion sharply alters the relative frequencies of all cell subtypes within the depleted sample relative to the original sample from which it is derived.

### 4. Computational Systems and Tools

Increasingly powerful machine learning tools are being developed to facilitate multi-omic analysis. Notably, BABEL (Wu et al, 2021) is a deep learning algorithm that computationally generates, from a single measured modality, other multiomic modalities in the same single cell. This allows researchers to perform downstream multiomic analysis at single-cell resolution as if joint profiling data had been collected. BABEL can accurately infer transcriptome-wide single-cell RNA profiles from genome-wide single-cell ATAC profiles, and vice versa. The BABEL architecture is composed of four modular neural networks as subcomponents. As described in Wu et al, two encoder networks are trained to project either RNA or ATAC profiles into a single, shared 16-dimensional latent representation. Similarly, two decoder neural networks are trained to take points in this shared latent representation and infer their corresponding RNA or ATAC profiles.

As methods for measuring different modalities of information within a cell become available, trade-offs between the number of modalities, depth of analysis, sample number, and cost are increasingly encountered. Once a class of samples has been jointly profiled by single-cell multiomic approaches, scientists can study future instances of such samples (in detailed time courses, perturbation, etc.) with the most economical or technically feasible modality, and infer the remaining information using BABEL. The BABEL neural network can facilitate single-cell analysis in clinical trials by limiting the number of omics modalities needed for sample analysis. The BABEL encoding architecture can be used to translate single-cell DNA Methyl-seq data to RNA-seq data, and additionally to translate DNA Methyl-seq data to single-cell chromatin accessibility M6A-MTase-seq (or ATAC-seq) data.

Generative pretrained models have been applied in various domains such as language and computer vision. Specifically, the combination of large-scale diverse datasets and pretrained transformers has emerged as a promising approach for developing foundation models. Parallels between language and cellular biology—in which texts contain words—similarly, cells are defined by regulatory DNA sequences, regulatory RNA sequences, and gene sequences. The model developed by Cui et al, (2024), called single-cell GPT, or scGPT, demonstrates the potential of the single-cell foundation model through three key aspects. First, scGPT represents a large-scale generative foundation model that allows transfer learning across a diverse range of downstream tasks. By achieving state-of-the-art performance on cell type annotation, genetic perturbation prediction, batch correction and multi-omic integration, the method showcases the effectiveness of the ‘pretraining universally, fine-tuning on demand’ approach as a generalist solution for computational applications in single-cell omics. Second, through the comparison of gene embeddings and attention weights between fine-tuned and raw pre-trained models, scGPT uncovers valuable biological insights into gene-gene interactions specific to various conditions, such as cell types and perturbation states. Third, and perhaps having the most dramatic long-term implications, the observations of Cui et al. (2024) reveal a scaling effect: larger pretraining data sizes yield superior pretrained embeddings and further lead to improved performance on downstream tasks.

The study of cell heterogeneity continues to be a key subject of interest in medical research. Understanding individual components of a heterogeneous cell population is of fundamental importance for progress in immunology research and for the emerging field of immune diagnostics. The growing trend to utilize single-cell molecular profiling strategies to avoid the pitfalls of analyzing bulk cell populations, unfortunately yields a blurred molecular profile of the “average” cell, and not the true profile of each individual cell. Currently existing methods encounter this pitfall particularly because the deep learning models are not properly trained. There is, therefore, a need for new analytical approaches capable of yielding molecular profiles of the cell regulatory states (e.g., chromatin regulatory states) of different cells in thousands of heterogeneous clinical samples, without the need to deploy single-cell analysis technologies at every step of the way. Such capabilities can be important when analyzing large numbers of stored clinical samples, such as frozen tissues or frozen white cells, which often are not of suitable quality for single-cell analysis. As mentioned previously, the way current models are trained cannot adequately perform this task.

To address the need for obtaining cell regulatory state information from complex tissues without the need to perform single-cell analysis of all available samples, the new DL models described herein have been uniquely trained. An initial analysis of a moderate-sized, representative subset of paired samples, containing two or more omics analysis modalities, is utilized to generate a training data set. In a non-limiting example, a first modality contains DNA methylation sequencing of bulk samples, and a second modality, performed on replicate samples of the same tissue, contains single-cell analysis. This bimodal analysis of paired samples is utilized only in the training stage of a large study. Availability of the dual modality omics data set provides for deployment of a trained or trained fine-tuned DL model that allows for additional biological samples to be subsequently analyzed only as bulk tissue, obviating the need for continued single-cell analysis of available samples. Accordingly, the unique improvements over traditional DL models are the more accurate development of the molecular profile and/or regulatory state of individual cells from bulk samples without the need for continued single-cell analysis of these available samples. In a non-limiting example, the uniquely trained DL models described herein can perform chromosome complement and cell-state deconvolution using deep DNA methylation sequencing data and single-cell data. “Bulk tissue” and “complex tissue” are used interchangeably, and refer to tissue that contains a heterogeneous population of cells. The heterogeneity in a population of cells can arise from different cell types, individual cells of the same type at different cell regulatory states, or a combination thereof. Preferably, heterogeneity in a population of cells arises from different cell types in the population of cells.

### REFERENCES

Bart A, et al., Direct detection of methylation in genomic DNA. Nucleic Acids Research 33(14): e124.
Beck M, et al., xLSTM: Extended Long Short-Term Memory. URL //arxiv.org/abs/2405.04517, 2024.
Brown et al., Language models are few-shot learners. Proceedings of NAACL-HLT 2019, pages 4171-4186 Minneapolis, Minnesota, Jun. 2-Jun. 7, 2019.
Cheung W A et al., Direct haplotype-resolved 5-base HiFi sequencing for genome-wide profiling of hypermethylation outliers in a rare disease cohort. 2023 Nature Communications 14: 3090.
Conti B A, et al., N6-methyladenosine in DNA promotes genome stability. Elife. 2025 Apr. 7; 13: RP101626.
Cui H, et al., scGPT: toward building a foundation model for single-cell multi-omics using generative AI. Nature Methods. 2024 Feb. 26.
Devlin et al., BERT: Pre-training of Deep Bidirectional Transformers for Language Understanding Proceedings of NAACL-HLT 2019, pages 4171-4186 Minneapolis, Minnesota, Jun. 2-Jun. 7, 2019.
Gu A, Dao T. Mamba: Linear-time sequence modeling with selective state spaces. arXiv preprint arXiv: 2312.00752v2, 2024.
Gupta P, et al., Advances in single-cell long-read sequencing technologies. NAR Genomics Bioinformatics 2024 May 20; 6(2).
Halliwell D O, et al., Double and single stranded detection of 5-methylcytosine and 5 hydroxymethylcytosine with OXFORD NANOPORE TECHNOLOGIES™ sequencing. Commun Biol. 2025 Feb. 15; 8(1): 243.
Iqbal W, Zhou W. Computational Methods for Single-cell DNA Methylome Analysis. Genomics Proteomics Bioinformatics. 2023 Feb; 21(1): 48-66.
Katoh K, Standley D M. MAFFT multiple sequence alignment software version 7: improvements in performance and usability. Mol Biol Evol. 2013 Apr.; 30(4): 772-80. doi: 10.1093/molbev/mst010. Epub 2013 Jan.16.
Kolmogorov, et al. Scalable OXFORD NANOPORE TECHNOLOGIES™ sequencing of human genomes provides a comprehensive view of haplotype-resolved variation and methylation. Nature Methods. 2023 Oct.; 20(10): 1483-1492.
Leduque B, Edera A, Vitte C, Quadrana L. Simultaneous profiling of chromatin accessibility and DNA methylation in complete plant genomes using long-read sequencing. Nucleic Acids Res. 2024 Jun. 24; 52(11): 6285-6297.
Li H. Minimap 2: pairwise alignment for nucleotide sequences. Bioinformatics. 2018 Sep. 15; 34(18): 3094-3100. available on the world wide web at //github.com/1h3/minimap2.
Li S et al., Comprehensive tissue deconvolution of cell-free DNA by deep learning for disease diagnosis and monitoring. 2023 PNAS 120(28): e2305236120.
de Lima L P, et al., CpGPT: a Foundation Model for DNA Methylation. 2024 bioRxivdoi. org/10.24.619766.
Luo, X, Kang, X, Schönhuth, A. Phasebook: haplotype-aware de novo assembly of diploid genomes from long reads. Genome Biol. 2021 vol 22(1): 299.
Marçais G, et al., MUMmer4: A fast and versatile genome alignment system. PLOS Comput Biol. 2018 Jan. 26; 14(1): e1005944.available at //github.com/mummer4/mummer.
Nguyen, et al. Sequence modeling and design from molecular to genome scale with Evo. Science. 2024 Nov. 15; 386(6723): eado9336. doi: 10.1126/science.ado9336.
Nguyen, et al. HyenaDNA: Long-Range Genomic Sequence Modeling at Single Nucleotide Resolution. ArXiv [Preprint]. 2023 Nov. 14: arXiv: 2306.15794v2.
Nguyen E et al., Sequence modeling and design from molecular to genome scale Evo. 2024 Science 386: 746.
Poli, M, et al., Hyena Hierarchy: Towards larger convolutional language models. arXiv preprint arXiv: 2302. 10866, 2023.
Ramani V, et al., Massively multiplex single-cell Hi-C. Nat Methods. 2017 Mar.; 14(3): 263-266.
Ramani V, et al., Sci-Hi-C: A single-cell Hi-C method for mapping 3D genome organization in large number of single cells. Methods. 2020 Jan. 1; 170: 61-68.
Schmidinger N, et al., Bio-xLSTM: Generative modeling, representation and in-context learning of biological and chemical sequences. arXiv preprint ar Xiv: 2411.04165, 2024.
Schuette G, Lao Z, Zhang B, ChromoGen: Diffusion model predicts single-cell chromatin conformations. Science Advances 2025 Nature Methods 11, eadr8265.

Sidorczuk K, et al., Genomic characterization of enterohaemolysin-encoding haemolytic Escherichia coli of animal and human origin. 2023 Microbial Genetics 9: 000999.

Szalata A, et al., Transformers in single-cell omics: a review and new perspectives. 2024 21: 1430.

Spix N J, et al., High-coverage allele-resolved single-cell DNA methylation profiling reveals cell lineage, X-inactivation state, and replication dynamics. Nat Commun. 2025 Jul. 8; 16(1): 6273.

Tayyebi, Z, Pine, A R, Leslie, C S. Scalable and unbiased sequence-informed embedding of single-cell ATAC-seq data with CellSpace. Nature Methods. 2024 Jun.; 21(6): 1014-1022. doi: 10.1038/s41592-024-02274-x. Epub 2024 May 9.
Tu X, et al. A combinatorial indexing strategy for low-cost epigenomic profiling of plant single cells. Plant Commun. 2022 Jul. 11; 3(4): 100308. doi: 10.1016/j. xplc.2022.100308. Epub 2022 Mar. 2.
Vaswani, et al., Attention is all you need. 31st Conference on Neural Information Processing Systems (NIPS 2017), Long Beach, CA, USA.
Vincent M et al., epiG: statistical inference and profiling of DNA methylation from whole-genome bisulfite sequencing data. 2017 Genome Biology 18: 38.
Vollger M R, et al. A haplotype-resolved view of human gene regulation. bioRxiv [Preprint]. 2025 Jun 2: 2024.06.14.599122.
Wu K E, et al. BABEL enables cross-modality translation between multiomic profiles at single-cell resolution. Proc Natl Acad Sci U S A. 2021 Apr. 13; 118.

1. A method comprising mapping single-cell-resolved DNA methylation sequence data to a set of epi-Haplotype Chromosomes (eHCs) to group the different eHCs in the set of eHCs into single-cell-resolved subsets of eHCs (epi-Haplotype Genomes), wherein each epi-Haplotype Genome (eHG) corresponds to the full chromosome complement of respective different single cells represented in the single-cell-resolved DNA methylation sequence data, wherein the single-cell-resolved DNA methylation sequence data are divided into sets of reads, wherein the reads in each set of reads correspond to respective different single cells, cell types, and/or cell regulatory states represented in the single-cell-resolved DNA methylation sequence data,
wherein the eHCs in the set of eHCs are each single-chromosome-resolved DNA methylation sequence data generated from assembly of long-read DNA methylation sequence data derived from one or more first sample(s),
wherein the single-cell-resolved DNA methylation sequence data are derived from one or more second sample(s),
wherein the long-read DNA methylation sequence data constitutes a first omics data set, wherein the single-cell-resolved DNA methylation sequence data constitutes a second omics data set,
wherein each first sample in the one or more first sample(s) constitutes a pair of samples with one of the second sample(s) in the one or more second sample(s),
wherein the first and second samples of a given pair of samples are derived from the same source and comprise overlapping cell types and/or cell lineages.

2. The method of claim 1, wherein the reads in each set of reads correspond to respective different cell regulatory states represented in the single-cell-resolved DNA methylation sequence data.

3. The method of claim 1, wherein the single-cell-resolved DNA methylation sequence data constitutes cell-regulatory-state-resolved DNA methylation sequence data.

4. The method of claim 1, wherein the first and second samples comprise samples from multiple cell types, multiple cell lineages, multiple tissues, and/or multiple organs from a single individual or from multiple individuals.

5. The method of claim 1, wherein the assembly of long reads of the long-read DNA methylation sequence data comprises alignment of the long reads comprising linearly contiguous DNA methylation information to form sets of continuously-aligned long reads.

6. The method of claim 5, wherein each set of continuously-aligned long reads that makes up a chromosome-identifying sub-chromosomal eHap constitute an eHC for that chromosome.

7. The method of claim 1, wherein the mapping of single-cell-resolved DNA methylation sequence data comprises scoring of sequence alignment between each eHC in the set of eHCs against single-cell-resolved reads in the second omics data set.

8. The method of claim 7, wherein the sequence alignment of single-cell-resolved reads containing repetitive DNA sequences are not scored.

9. The method of claim 7, wherein the mapping of single-cell-resolved DNA methylation sequence data comprises alignment of a pool of DNA methylation sequence data from the first omics data set with DNA methylation sequence data from the second omics data set,
wherein the pool of DNA methylation sequence data comprises one or more of the eHCs,
wherein the DNA methylation sequence data from the second omics data set comprises one or more sets of single-cell-resolved reads, wherein the single-cell-resolved reads in a given set of single-cell-resolved reads are derived from an individual cell, cell type, and/or cell regulatory state, and
wherein DNA methylation sequence data from the second omics data set comprises defined genomic coordinates for the single-cell-resolved reads in the set(s) of single-cell-resolved reads except for the single-cell-resolved reads containing repetitive DNA sequences.

10. The method of claim 9, wherein the single-cell-resolved reads in a given set of single-cell-resolved reads are derived from:
(i) a cell regulatory state; and/or
(ii) a single cell.

11. (canceled)

12. The method of claim 7, wherein the assembly of long-read DNA methylation sequence data further comprises storing the eHCs in a first structured data format, wherein the mapping of single-cell DNA methylation sequence data further comprises storing the eHGs in a second structured data format such as a second array, and
wherein DNA methylation sequence data from both the first and second omics data sets are mapped to the same eHGs except that DNA methylation sequence data from the second omics data set containing repetitive DNA sequences are not mapped.

13. The method of claim 12, wherein:
(i) the first structured data format is a first array; and/or
(ii) the second structured data format is a second array.

14. The method of claim 13, wherein:
(a) the first array is a first matrix; and/or
(b) the second array is a second matrix.

15-16. (canceled)

17. The method of claim 14, wherein the second matrix comprises the same structure and dimensions as the first matrix.

18. The method of claim 1, wherein the mapping of single-cell-resolved DNA methylation sequence data comprises alignment of single-cell-resolved reads in the second omics data set with methylated DNA nucleotides in the eHCs except for the single-cell-resolved reads containing repetitive DNA sequences, wherein the alignment comprises:
aligning single-cell-resolved reads from the second omics data set with each of the eHCs except for the single-cell-resolved reads containing repetitive DNA sequences; and
scoring the sequence alignment of the aligned single-cell-resolved reads with the eHCs.

19. The method of claim 18, wherein the grouping of eHCs into the eHGs comprises:
selecting the eHC yielding the best aggregate sequence alignment score for each aligned class of single-cell-resolved reads, and grouping the best scoring eHCs into a set of eHCs that constitute the eHG for the aligned class of single-cell-resolved reads.

20. The method of claim 19, wherein:
(i) the aligned class of single-cell-resolved reads corresponds to a single cell regulatory state; or
(ii) the aligned class of single-cell-resolved reads corresponds to a single cell regulatory state and to multiple single cells representing that cell regulatory state; or
(iii) the class of single-cell-resolved reads corresponds to a single cell regulatory state; or
(iv) the different aligned classes of single-cell-resolved reads constitute cell-regulatory-state-resolved reads.

21-23. (canceled)

24. The method of claim 18, wherein segments of eHCs to which single-cell-resolved reads containing repetitive DNA sequences were not aligned are assigned as the locus-specific DNA methylation states for those segments, thereby assigning to the corresponding single-cell-resolved data set the locus-specific DNA methylation states for all repetitive DNA sequences in the eHCs.

25. The method of claim 24, wherein the assigning further comprises storing a combination of assigned single-cell-resolved reads and assigned segments of eHC in a third structured data format,
wherein the third structured data format comprises long-read DNA methylation sequence data from the first omics data set(s) and corresponding single-cell-resolved DNA methylation sequence data from the second omics data set,
wherein each assigned segment of eHC is stored in a position in the third structured data format that corresponds to the single-cell-resolved DNA methylation sequence data.

26. The method of claim 25, wherein the third structured data format is a third array,
optionally wherein the third array is a third matrix.

27. (canceled)

28. The method of claim 1, wherein each first sample comprises a first portion of a biological sample of cells and/or tissue obtained from a subject and each second sample comprises a second portion of the same biological sample of cells and/or tissue obtained from the subject.

29. The method of claim 28, wherein the biological sample from which each pair of samples are taken is different for each pair of samples.

30-32. (canceled)

33. The method of claim 1, wherein:
(a) the long reads of the long-read DNA methylation sequence data in the first omics data set comprise about 3,000 to about 100,000 nucleotides, inclusive; or
(b) the single-cell-resolved reads of the single-cell-resolved DNA methylation sequence data in the second omics data set comprise about 50 to about 600 nucleotides, inclusive; or
(c) the first omics data set comprises sequencing data with a depth of coverage of more than 300, or more than 600, for each nucleotide position in the genome that is sequenced.

34-35. (canceled)

36. The method of claim 1, wherein the first and/or second omics data set(s) comprise M5C modification data; or M6A modification; or M5C modification data and sequences that include bases with M6A modifications data, optionally wherein:
(i) the M5C modification data comprises a complete data set for all methylcytosines within each read in the first and/or second omics data set(s); or
(ii) the M6A modification data comprises a complete data set for all methyladenosines within each read in the first and/or second omics data set(s); or
(iii) the M5C modification data and sequences that include bases with M6A modifications comprises a complete data set for all methylcytosines and all methyladenosines within each read,
optionally wherein the first and/or second omics data set(s) further include 5-hydroxy-methylCytosine modifications.

37-40. (canceled)

41. The method of claim 1, wherein the second omics data set is generated by single-cell Methyl-seq.

42-44. (canceled)

45. The method of claim 1, wherein each eHG corresponds to the full chromosome complement of a cell regulatory state represented in the single-cell-resolved DNA methylation sequence data.

46. The method of claim 45, wherein the single-cell-resolved DNA methylation sequence data constitutes cell-regulatory-state-resolved DNA methylation sequence data.

47. The method of claim 45, wherein the full chromosome complement of each cell regulatory state comprises repetitive DNA sequences that are differentially methylated in stressed, drug-treated, or diseased cells, and
wherein the cell regulatory states comprise one or more disease states.

48. The method of claim 1, wherein, prior to production of the first and second omics data sets, the first and second samples or their source sample are depleted of the most highly abundant cell subtypes, wherein the first and second sample(s) are depleted first and second samples.

49. The method of claim 1, wherein one or more additional first and second omics data sets are produced from one or more additional first and second sample derived from the same source as the first and second samples of a given pair of samples, wherein the additional first and second samples or their source sample are depleted of the most highly abundant cell subtypes, wherein the additional first and second samples are depleted first and second samples.

50. The method of claim 48, wherein long-read DNA methylation sequence data derived from one or more depleted first samples result in a larger proportion of the eHCs being derived from cell types and/or cell lineages present at low frequency in undepleted samples as compared to when long-read DNA methylation sequence data derived from one or more undepleted first samples are assembled into eHCs, and
wherein single-cell-resolved DNA methylation sequence data derived from one or more depleted second samples result in a larger proportion of the eHCs being derived from cell types and/or cell lineages present at low frequency in undepleted samples as compared to when single-cell-resolved DNA methylation sequence data derived from one or more undepleted second samples are aligned to eHCs to group the eHCs into eHGs.

51. The method of claim 1, further comprising formulating the eHCs as a training set for a machine learning architecture,
wherein the architecture comprises a foundation model and a transformer,
wherein the model and transformer each comprise encoding systems for training based on the training set of eHCs and corresponding second omics data sets, and
wherein the model and transformer learn the sequence relationships among all of the eHGs,
optionally further comprising generating, using the machine learning architecture, one or more further eHCs from one or more further omics data set(s) that each comprise further long-read DNA methylation sequence data derived from one or more further sample(s),
wherein the machine learning architecture generates a plurality of eHGs each corresponding to the full chromosome complement of respective different single cells represented in the further long-read DNA methylation sequence data of the further omics data set(s), wherein each eHG represents a cell regulatory state for a single cell in the further sample(s).

52. (canceled)

53. A method comprising analyzing a test omics data set derived from a test sample using a deep learning (DL) model, wherein the DL model has been trained on one or more first omics data set(s) from one or more first sample(s), and one or more second omics data set(s) from one or more second sample(s),
wherein each first sample in the one or more first sample(s) constitutes a pair of samples with one of the second sample(s) in the one or more second sample(s),
wherein the first and second samples of a given pair of samples are derived from the same source and comprise overlapping cell types and/or cell lineages,
wherein the test sample is derived from the same type of source as the source of at least one of the pairs of first and second samples;
assigning cell regulatory states to cells in the test sample without a need for analyzing single-cell omics data from the test sample; and
displaying the assigned cell regulatory states on a graphical user interface in real-time, such as within 1, 2, 3, 4, 5, 10, 15, 20 minutes, or no more than 30 minutes after receiving the eHC-assembled test omics data set.

54-84. (canceled)
